(19) United States
(12) Patent Application Publication
(10) Pub. No.: US 2019/0374334 A1
(43) Pub. Date: Dec. 12, 2019

(54) INTRAOCULAR LENS DEVICE AND RELATED METHODS

(71) Applicant: LensGen, Inc., Irvine, CA (US)

(72) Inventors: Daniel Brady, San Juan Capistrano, CA (US); Thomas Silvestrini, Alamo, CA (US); Ramgopal Rao, Irvine, CA (US)

(21) Appl. No.: 16/434,026

(22) Filed: Jun. 6, 2019

Related U.S. Application Data
(60) Provisional application No. 62/682,037, filed on Jun. 7, 2018.

(51) Int. Cl. A61F 2/16

(52) U.S. Cl. CPC A61F 2/1662; A61F 2/1618; A61F 2/1645; A61F 2240/001; A61F 2250/0097; A61F 2002/1696; A61F 2250/0087

(57) ABSTRACT

An intraocular device that includes a base member is provided. The device can be an accommodation intraocular lens device with the base member and a power changing lens. The base member comprises an annular haptic that surrounds a central cavity having an open end. The power changing lens is configured to fit within the central cavity. The haptic comprises one or more projections, e.g., tabs that hold another device in position. In the case of the accommodating intraocular lens device, the other device is the power changing lens. The base member and the power changing lens are maintained separate until assembly in the eye of the patient. During assembly, the base member is advanced into the capsular bag of a patient through a capsulorhexis and oriented such that the open end of the central cavity faces the cornea. Subsequently, the power changing lens is advanced into the central cavity through the capsulorhexis. The one or more tabs are placed anterior of the power changing lens to secure the power changing lens within the cavity.

### INCORPORATION BY REFERENCE TO ANY PRIORITY APPLICATIONS

Any and all applications for which a foreign or domestic priority claim is identified in the Application Data Sheet as filed with the present application, including U.S. Application No. 62/682,037, filed on Jun. 7, 2018, are hereby incorporated by reference under 37 CFR 1.57. In addition, this application incorporates by reference the entirety of each of the following patent applications: U.S. application Ser. No. 15/144,544 filed on May 2, 2016, issued as U.S. Pat. No. 10,159,564; U.S. application Ser. No. 14/447,621 filed on Jul. 31, 2014, issued as U.S. Pat. No. 10,004,596; and International Application No. PCT/US2016/064491 filed on Dec. 1, 2016 and published as WO 2017/096087.

### BACKGROUND OF THE INVENTION

### Field of the Invention

This application relates to an intraocular device configured to be placed in a capsular bag of a human eye following a capsulotomy to hold the capsular bag open and to provide a cavity in which an accommodating intraocular device can be placed, and to systems and methods for implanting the same.

### Description of the Related Art

Surgical procedures on the eye have been on the rise as technological advances permit for sophisticated interventions to address a wide variety of ophthalmic conditions. Patient acceptance has increased over the last twenty years as such procedures have proven to be generally safe and to produce results that significantly improve patient quality of life.

Cataract surgery remains one of the most common surgical procedures, with over 28 million cataract procedures being performed worldwide per year. It is expected that this number will continue to increase as average life expectancies continue to rise. Cataracts are typically treated by removing the crystalline lens from the eye and implanting an intraocular lens (“IOL”) in its place. As conventional IOL devices are designed to provide clear distance visions, they fail to correct for presbyopia. As a result, reading glasses are still required. Thus, although the vision of patients who undergo a standard IOL implantation will not be clouded by the cataract, they are unable change focus from far to near.

Furthermore, it is unfortunately common for the IOL to settle into a position other than what was expected or planned prior to or during the surgery. Even if intraoperative measurements are made to confirm the optics during the procedure, the position of the IOL can change following surgery due to a number of processes. For instance, traditional IOLs are low volume structures optimized for insertion through small incisions. As such traditional IOLs are thin in the anterior-posterior direction, allowing anterior and posterior aspects of the capsular bag to come together. When adjacent layers of the anterior and posterior aspects of capsular bag contact, a process called fibrosis occurs, which can change the IOL position or orientation and/or can lead to posterior capsular opacification.

### SUMMARY OF THE INVENTION

Accordingly, there is a need for an intraocular device that can be placed in the capsular bag following capsulotomy and can provide enhanced outcomes for patient. Enhanced outcomes can be in a variety of forms. For instance accommodating IOL device can be assembled in the eye, which device can have the ability to change the power of the eye for focusing on near, far, and in-between. Another enhanced outcome made possible by the devices and methods disclosed herein is the ability to select a lens that corrects astigmatism and other higher order aberrations. The devices and method disclosed herein are uniquely configured to assure rotational position of the aberration correcting optic. A further enhanced outcome made possible by the devices and methods disclosed herein is providing assurances of proper lens positioning in surgery and thereafter. This is made possible by the configuration of a base member which is positioned in the eye during the surgery in a manner that reduce, minimizes, or eliminates posterior-anterior drift following placement so that the patient's vision is substantially unchanged following the surgery.

In various embodiments, a base member for an accommodating intraocular lens device is provided. The base member includes a base lens and a haptic. The haptic includes a first open end, a second end coupled with the base lens, and an outer periphery configured to engage an equatorial region of a capsular bag. The haptic further comprises an inner periphery and a height between a first edge and a second edge. The inner periphery is disposed about a cavity and having a lens retention portion configured to receive and retain a power changing lens.

In some embodiments, a portion of the haptic anterior to the lens retention portion of the cavity can comprise enhanced flexibility compared to the lens retention portion. In some other embodiments, a portion of the haptic disposed about the lens retention portion of the cavity can comprise enhanced stiffness compared to the lens retention portion. The haptic can comprise a plurality of compression members. Each compression member can comprise a circumferentially extending contact zone, a first radial force member coupled with a first circumferential end and a second radial force member coupled with a second circumferential end of each contact z one.

Various embodiments of the accommodating intraocular lens device can further comprise a compression member hinge disposed between each first radial force member and each second radial force member. The compression member hinge can comprise a groove disposed in the outer periphery of the haptic. The groove can comprise an anterior portion that extends entirely from the outer periphery to the inner surface of the haptic and a posterior portion that is enclosed by the contact zone. The anterior portion can extend about 35% of the height of the haptic from the first open end. The anterior portion of the compression member hinge can be more circumferentially deformable than the posterior portion.

Various embodiments of the accommodating intraocular lens device can comprise a plurality of contact zones, each contact zone disposed between adjacent internal hinges formed in the inner surface of the haptic. Various embodiments of the accommodating intraocular lens device can further comprise a plurality of external hinges disposed about the outer periphery of the haptic. The external hinges can be spaced circumferentially from the internal hinges. The external hinges can comprise a groove extending radially inwardly from the outer periphery. The internal hinges can comprise a groove extending radially outwardly from the inner surface. The radially inner-most edge of the grooves of the external hinges can be radially inward of the radially outer-most edge of the grooves of the internal hinges.

Various embodiments of the accommodating intraocular lens device can comprise a plurality of contact zones. Alternating contact zones can be spaced apart from and not coupled with the base lens radially inwardly thereof. Various embodiments of the accommodating intraocular lens device can further comprise a plurality of spaced apart radial hinges comprising a first portion coupled with the base lens and a second portion coupled with the haptic. In various embodiments of the accommodating intraocular lens device, the second end of the haptic can comprise a ring and a hinge having a first end coupled to the ring and a second end coupled to the inner surface of the haptic.

In various embodiments of the accommodating intraocular lens device, the base lens can comprise a haptic interface surface and the ring of the haptic can comprise a lens interface surface. The haptic interface surface of the base lens can be coupled to the lens interface surface of the ring of the haptic. The haptic can comprise a first material configured to transfer force between outer periphery and the inner surface and the base lens can comprise a second material different from the first material. The lens retention portion can comprise a retention member comprising an anterior surface having a ridge formed thereon on an anterior side thereof. The ridge can be visible during implantation to enable a visual confirmation of proper placement of a power changing lens posterior to the retention member.

Various embodiments of an accommodating IOL can comprise the accommodating intraocular lens device described herein and a power changing lens configured to fit within the cavity. The power lens can comprise a first side, a second side, a peripheral portion coupling the first and second sides, and a closed cavity configured to house a fluid. The first side of the power changing lens can be spaced from the first edge of the haptic. In various embodiments, at least a portion of the haptic can comprise a material with high contrast to the material of the peripheral portion of the power changing lens. In some embodiments, at least a portion of the haptic can comprise an at least partially opaque dye and the peripheral portion of the power changing lens is translucent. In some other embodiments, at least a portion of the haptic can comprise a first color surface and the peripheral portion of the power changing lens can comprise a second color surface visually distinct from the first color surface. In some embodiments, at least a portion of the haptic can comprise a first visual pattern and the peripheral portion of the power changing lens can comprise a second visual pattern visually distinct from the first visual pattern.

Various embodiments of the accommodating IOL can comprise a plurality of open channels extending from outside of the accommodating IOL to a space between the base lens and the second side of the power changing lens. In some embodiments of the accommodating IOL, the haptic can comprise a plurality of spaced apart radial hinges comprising a first portion coupled with the base lens and a second portion coupled with the haptic, a gap provided between the radial hinges and the second side of the power changing lens when the accommodating intraocular lens is in an accommodated state and when the accommodating intraocular lens is in a disaccommodated state.

In another embodiment, a method of assembling an intraocular lens in a capsular bag of an eye of a patient is provided. An injector barrel is advanced into the eye of the patient. The injector barrel contains a base member. The base member has a base lens and a ring-shaped member coupled to the base lens. In various embodiments, the ring-shaped member can be configured as a haptic. The ring-shaped member has a first edge located at an open end thereof. The first edge is disposed about an anterior end of a cavity. The base lens is coupled with a second edge of the ring-shaped member. The base member is folded about a transverse axis of the base lens or of a portion of the ring-shaped member or the haptic such that the cavity is on a concave side of the transverse axis and the base lens is on a convex side of the transverse axis. The injector barrel is oriented such that the concave side of the base member fold is oriented anteriorly relative to the patient's eye. The base member is advanced out of the injector barrel such that the base member unfolds with the base lens facing posteriorly toward the posterior surface of the capsular bag and the cavity facing anteriorly toward the cornea. A power changing lens is advanced into the cavity of the base member within the capsular bag of the eye of the patient. The power changing lens has an anterior surface, a posterior surface, and a circumferential portion disposed between the anterior surface and the posterior surface. The power changing lens is folded about a transverse axis of the power changing lens. The power changing lens is unfolded within the cavity of the base member such that the circumferential portion of the power changing lens engages a side of the ring-shaped member facing the cavity.

In one variation the posterior surface of the power changing lens is disposed on a concave side of a power changing lens fold (e.g., concave side of the transverse axis of power changing lens) and the anterior surface is disposed on a convex side of the power changing lens fold (e.g., concave side of the transverse axis of power changing lens). The power changing lens is oriented such that the concave side of the power changing lens fold faces posteriorly prior to unfolding.

In another variation, the power changing lens is folded such that the posterior surface is disposed on a convex side of a power changing lens fold and a deformable membrane on the anterior surface is disposed on a concave side of the power changing lens fold further comprising orienting the power changing lens such that the concave side of the folded power changing lens faces anteriorly.

In another embodiment, a method of assembling an intraocular lens in a capsular bag of an eye of a patient is provided. A bowl-shaped member is positioned in a capsular bag of an eye with a base lens of the bowl shaped member contacting a posterior inside surface of the capsular bag. A haptic contacts an equatorial region of the capsular bag. The haptic has a first edge disposed forward of an anterior surface of the base lens. The bowl-shaped member defines a cavity therein. A power changing lens is advanced into the cavity of the bowl shaped member in a folded state wherein opposing sides of a circumferential portion of the power changing lens are brought together. The power changing lens is unfolded within the cavity of the bowl-shaped base member such that the circumferential portion of the power changing lens is retained within the haptic.

In one variation, the power changing lens is unfolded while the concave side of the fold faces posteriorly. In another variation, a deformable membrane of the power changing lens faces a concave side of the fold, wherein the fold faces anteriorly.

An innovative aspect of the subject matter of this application is embodied in an ophthalmic lens system, comprising an injector comprising a plunger and a barrel having a lumen extending proximally from a distal end along a longitudinal axis; a base member comprising a base lens and a ring-shaped member coupled to the base lens, the ring-shaped member comprising a first edge defining an open end of the base member, the base lens being coupled with a second edge of the ring-shaped member, the base member being folded and disposed in the lumen of the barrel of the injector such that the base lens is adjacent to the lumen and the cavity is disposed between the base lens and the longitudinal axis of the lumen; and a power changing lens comprising an anterior surface, and a posterior surface coupled with the anterior surface, the power changing lens being folded such a circumferential portion disposed between the posterior surface and the anterior surface is brought together. The power changing lens is disposed in the lumen of the barrel proximal to the base member.

In various embodiments of the ophthalmic lens system, the base lens and the anterior surface can be disposed on opposite sides of the lumen of the barrel of the injector. The anterior surface of the power changing lens can comprise a flexible membrane and the posterior surface of the power changing lens can comprise a surface of powered lens. A fluid can be contained between the anterior surface of the power changing lens and posterior surface of the power changing lens. The power changing lens and the base member can be separated from and not connected to each other. The anterior surface can be disposed adjacent to the lumen and the posterior surface can be disposed between the anterior surface and the longitudinal axis of the lumen. The posterior surface can be disposed adjacent to the lumen and the anterior surface is disposed between the posterior surface and the longitudinal axis of the lumen.

In some embodiments an intraocular lens component is provided that includes an anterior side, a posterior side, a peripheral portion and a visible color structure. The anterior side has an anterior optical surface disposed across an optical axis of the lens component. The posterior side has a posterior optical surface disposed across the optical axis. The peripheral portion has an anterior portion coupled to the anterior side and a posterior portion coupled to the posterior side. The peripheral portion couples the anterior side to the posterior side of the intraocular lens component. The visible color structure is disposed in the peripheral portion between the anterior portion and the posterior portion thereof.

In another embodiment, an intraocular lens component is provided that includes an anterior side, a posterior side, a peripheral portion and a rotational position feature. The anterior side has an anterior optical surface disposed across an optical axis of the lens component. The posterior side has a posterior optical surface disposed across the optical axis. The peripheral portion has an anterior portion coupled to the anterior side and a posterior portion coupled to the posterior side. The peripheral portion couples the anterior side to the posterior side of the intraocular lens component. The rotational position feature is disposed on or in the peripheral portion and is configured to provide simultaneous confirmation of orientation about at least two axes.

In another embodiment a method of assembling a base member of an intraocular lens is provided. The base member haptic is provided. The base member haptic has a first open end, a second end opposite the first open end, and an outer periphery configured to engage an equatorial region of a capsular bag. The second end has a lens interface portion. A base lens a central optical portion and a peripheral haptic interface portion is provided. The haptic interface portion of the base lens is coupled with the lens interface portion of the base member haptic. The base lens and the base member haptic are secured together at the lens interface portion and the haptic interface portion.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1 shows an anterior perspective, broken out view of an eye with an accommodating intraocular lens (IOL) device according to one embodiment of the present disclosure disposed therein;

FIG. 2 is an anterior perspective view of the accommodating IOL device shown in FIG. 1;

FIG. 2A and FIG. 2A-1 are anterior views of the accommodating IOL device of FIG. 2;

FIG. 2B is an exploded view of the accommodating IOL device of FIG. 2;

FIG. 2C is a cross-sectional view of the accommodating IOL device of FIG. 2 taken at section plane 2C-2C;

FIG. 2D is a cross-sectional view of the accommodating IOL device of FIG. 2 taken at section plane 2D-2D;

FIG. 3A is an anterior perspective view of a base member of an ophthalmic device, such as the accommodating IOL device of FIG. 2;

FIG. 3B is an anterior view of the base member of FIG. 3A;

FIG. 3C is a cross-section view of the base member of FIG. 3A taken at section plane 3C-3C in FIG. 3B;

FIG. 4A is a perspective view of a power changing lens of the accommodating IOL of FIG. 2;

FIG. 4B is a cross-sectional view of the power changing lens of FIG. 4A taken at section plane 4B-4B;

FIG. 4C is an anterior view of the power changing lens of FIG. 4A with a rotational position feature indicating simultaneous visual confirmation of orientation.

FIG. 4D is a posterior view of the power changing lens of FIG. 4C.

FIG. 4E is an anterior perspective view of a non-accommodating lens.

FIG. 4F is an anterior perspective view of an extended depth of focus (EDOF)/trifocal lens.

FIG. 5 is a detail view of a portion of the base member of FIG. 3 at detail 5-5 in FIG. 3B;

FIG. 5A is a cross-section at section plane 5A-5A in the detail view of FIG. 5;

FIG. 6 is a detail view of a portion of the base member of FIG. 3 at detail 6-6 in FIG. 3B;

FIG. 6A is a cross-section at section plane 6A-6A in the detail view of FIG. 6;

FIG. 7 is a detail view of a portion of the base member of FIG. 3 at detail 7-7 in FIG. 3B;

FIG. 7A is a cross-section at section plane 7A-7A in the detail view of FIG. 7;

FIG. 8 is an anterior view similar to that of FIG. 3B showing a modified embodiment of a retention member aiding a surgeon assembling an accommodating IOL similar to that of FIG. 1 in the eye;

FIG. 8A is a cross-section at section plane 8A-8A in the detail view of FIG. 8;

FIG. 8B and FIG. 8C show the visual indication provided by the modified embodiment of a retention member to aid a surgeon in correctly assembling the accommodating IOL similar to that of FIG. 1 in the eye;

FIG. 9 is a top view similar to that of FIG. 3B showing a modified embodiment of a ring member visually aiding a surgeon assembling an accommodating IOL similar to that of FIG. 1 in the eye;

FIG. 10A schematically illustrates an unaltered human eye, which may be in need of cataract or presbyopia treatment surgery;

FIG. 10B schematically illustrates the human eye following removal of the contents of the crystalline lens, leaving the capsular bag intact;

FIG. 10C shows delivery of a base member lens into the eye following removal of the crystalline lens, the base member being folded during delivery;

FIG. 10D shows the base member being un-folded within the capsular bag;

FIG. 10E shows the base member completely unfolded within the capsular bag;

FIG. 10F shows a power changing lens being delivered into the eye through the same incision used to deliver the base member in FIG. 10C;

FIG. 10E-1 schematically shows a first approach for delivering a power changing lens opposite to a retention member of the base member of FIG. 3;

FIG. 10E-2 schematically shows a second approach for delivering a power changing lens opposite to a retention member of the base member of FIG. 3A;

FIG. 10G shows the power changing lens according to the first approach being advanced out of an injector barrel into the base member;

FIG. 10H shows the power changing lens in the process of being unfolded and disposed under the retention members of the base member of FIG. 3A;

FIG. 10I shows the power changing lens, with the rotational position feature of FIG. 4C, under the retention members of the base member of FIG. 3A; and

FIG. 11 shows a system including an injector having a base member disposed in a distal portion thereof and a power changing lens disposed in a proximal portion thereof.

### DETAILED DESCRIPTION OF THE PREFERRED EMBODIMENT

This application discloses a base member 102 for a multi-component IOL device 100. The components of the IOL device are maintained separate until being assembled within an eye 50 of the patient. Once assembled, the IOL device 100 can provide premium lens performance. In a first embodiment, discussed in SECTION I, the IOL device 100 is configured to provide accommodation when subject to ocular forces and thus will be referred to herein as an accommodating IOL device 100. Various embodiments of the IOL device 100 can be configured to correct for a higher order aberration, such as astigmatism, with or without additionally providing accommodation. Furthermore, various embodiments of the IOL device 100 can be configured to provide post-surgical emmetropia, with or without accommodation, using the base member as a volume restoring member.

### I. Accommodating IOL Device Embodiments

Providing clear focus over distances from near to far is one of the chief aims of surgery in the front part of the eye. An accommodating IOL device 100 described herein in various embodiments is uniquely configured for this objective.

### A. Eye Anatomy and Accommodation

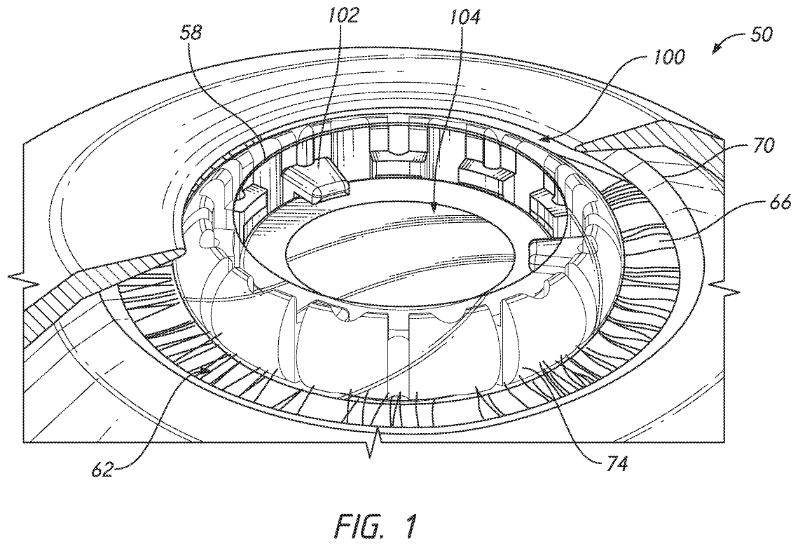

FIG. 1 shows an eye 50 following placement of the accommodating IOL device 100. The natural lens of the eye 50 has been modified by a capsulotomy procedure in which an opening 58 is formed in the natural crystalline lens the capsular bag 62 is evacuated of its contents through the opening 58. The opening 58 provides access through an access pathway from an exterior of the eye 50 for placement of the accommodating IOL device 100 in the capsular bag 62.

An equatorial region 74 of the capsular bag 62 is coupled by zonules 66 to a ciliary muscle 70. The zonules 66 are connective tissues that can stretch the capsular bag 62. When the ciliary muscle 70 is in a rest state the zonules 66 are stretched and apply a tension force to the capsular bag 62. When the eye 50 attempts to accommodate, the ciliary muscle 70 contracts, reducing the tension in the zonules 66. These accommodation processes result in a compression force on the base member 102 as discussed further below. Without being bound to a particular theory, it is believed that the capsular bag 62 contracts when the tension in the zonules 66 is reduced. That contraction applies a compression force the base member 102 to cause accommodation of a power changing lens 104 that can be placed in the base member 102. The ocular forces of the eye 50 are sufficient to change the shape of one or more optical surface of the power changing lens 104, resulting in accommodation.

### B. Separate Component Accommodating IOL Structure

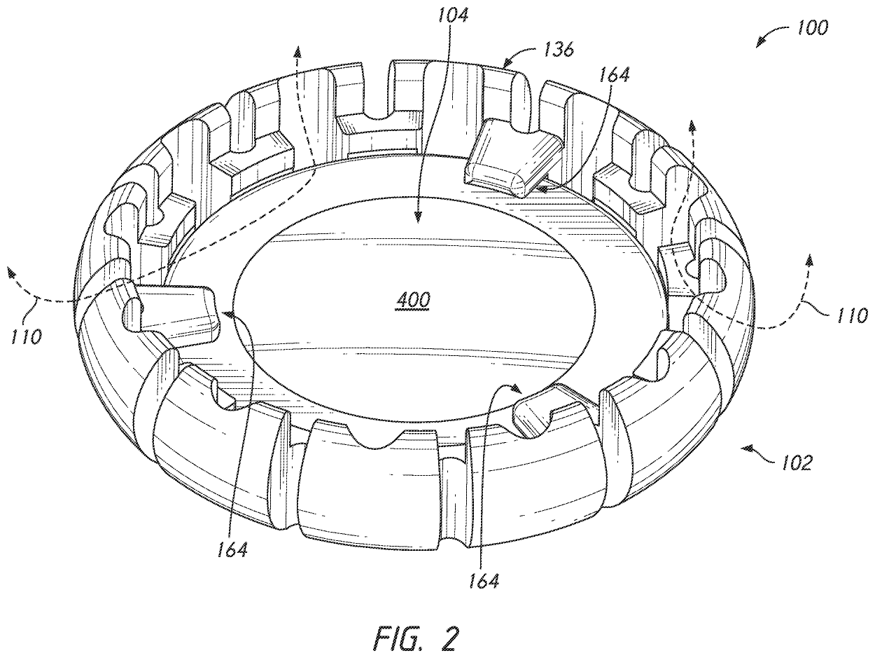

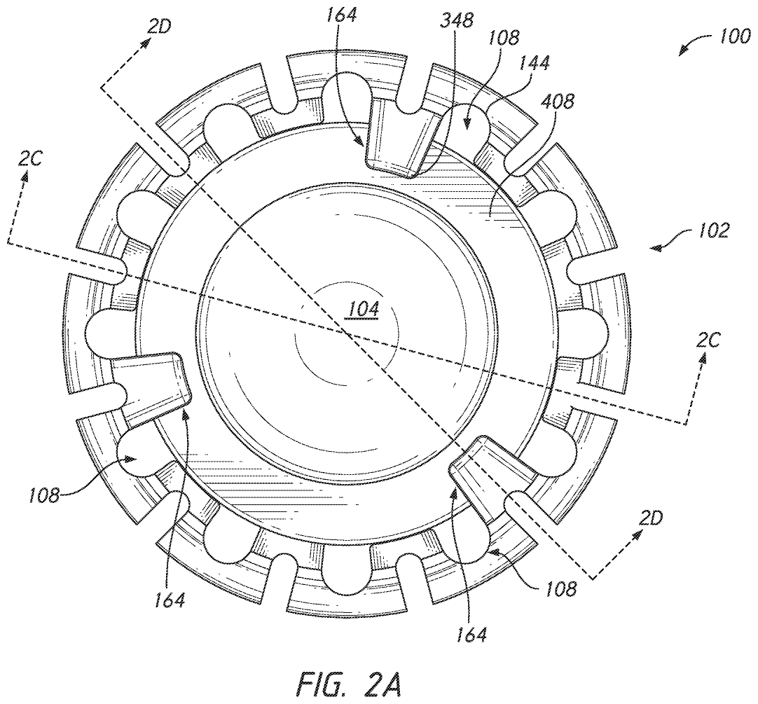

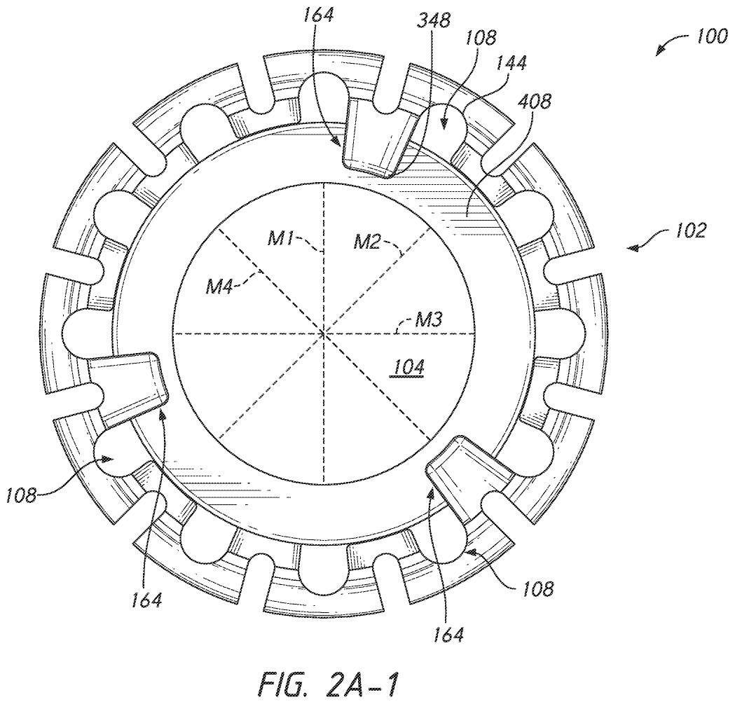

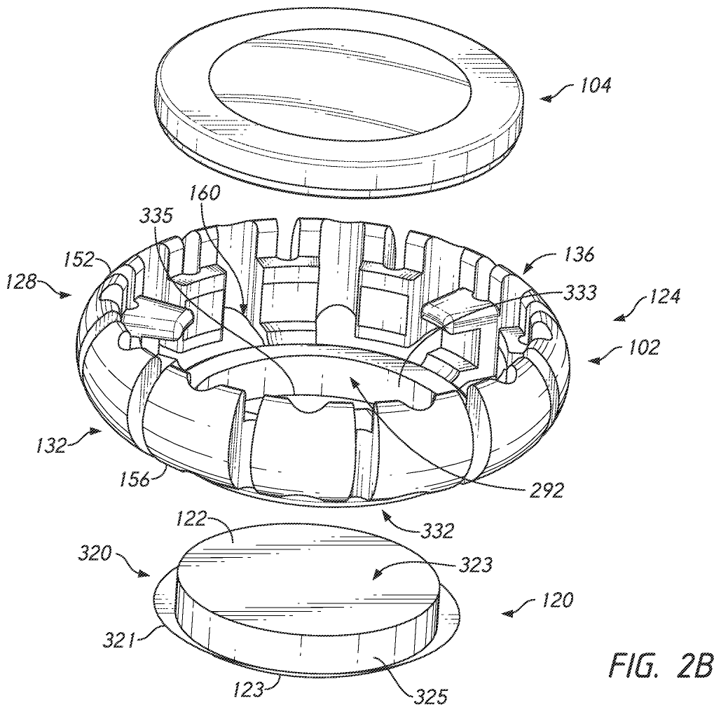

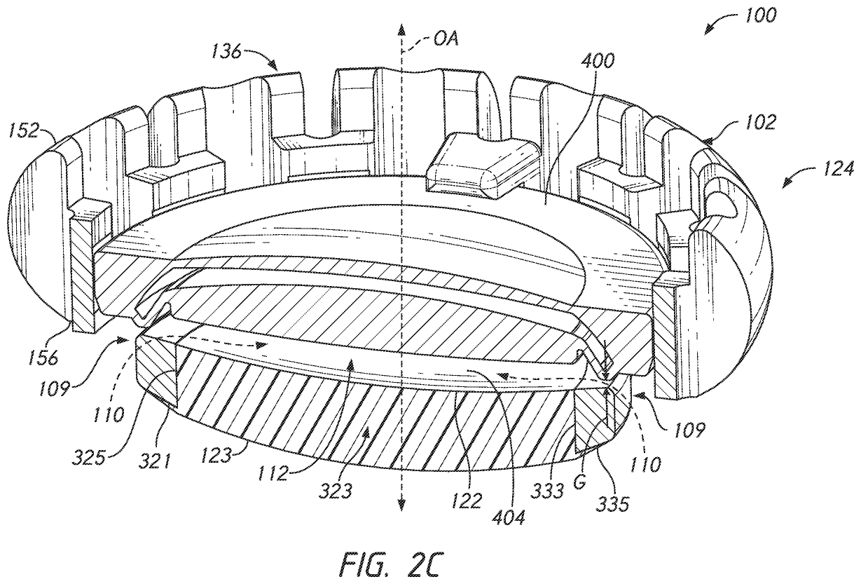

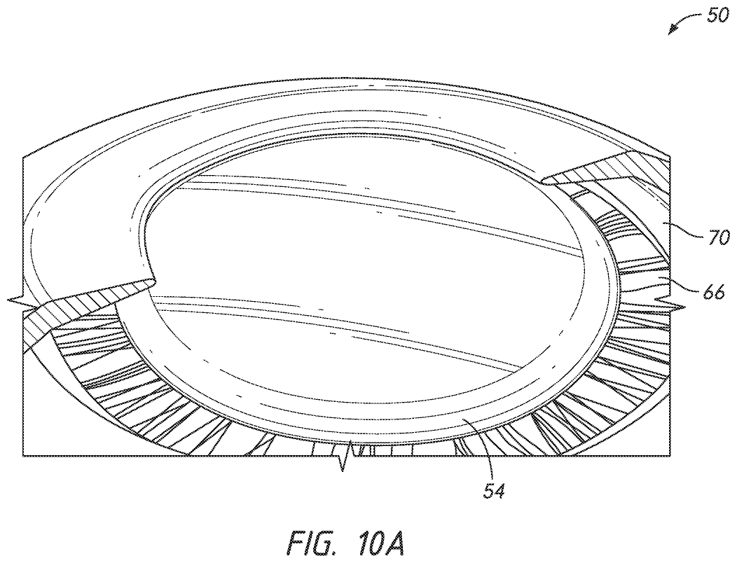

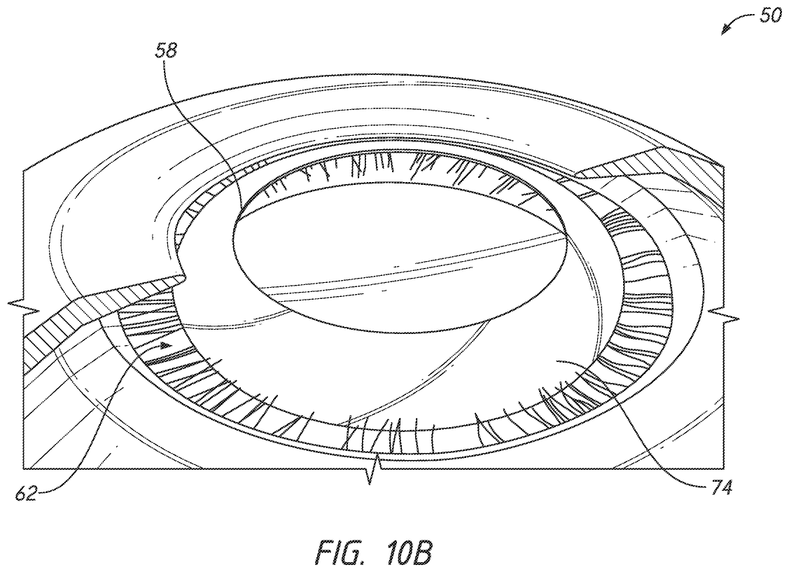

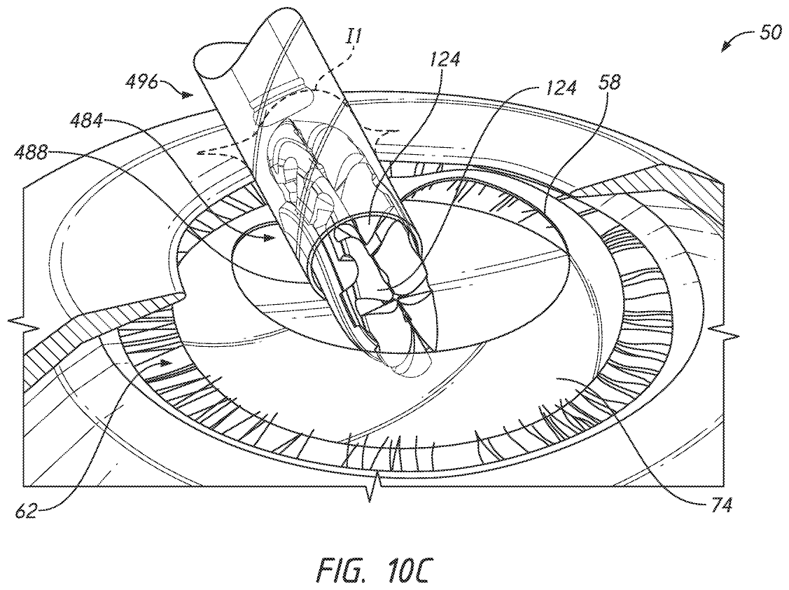

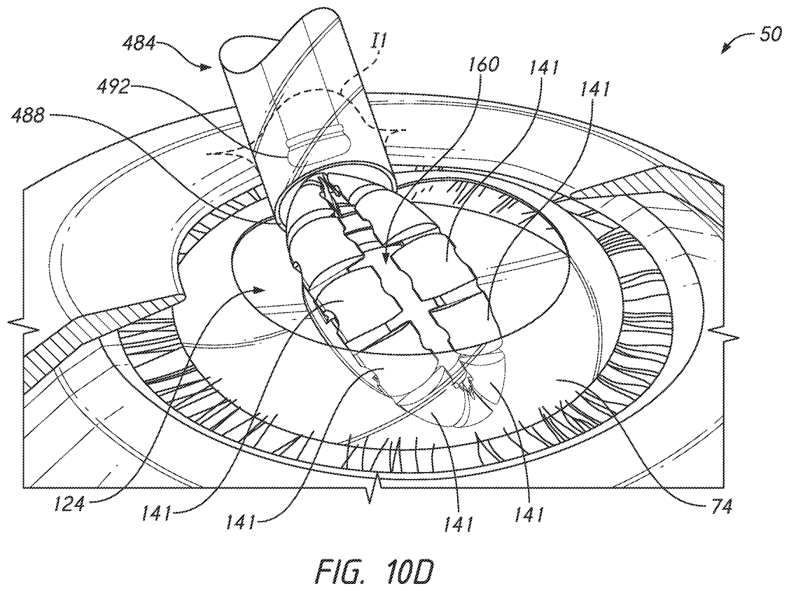

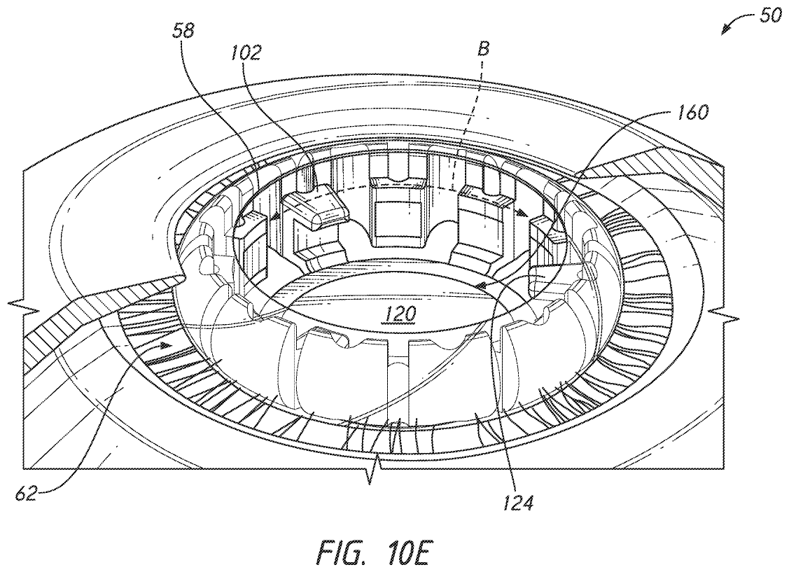

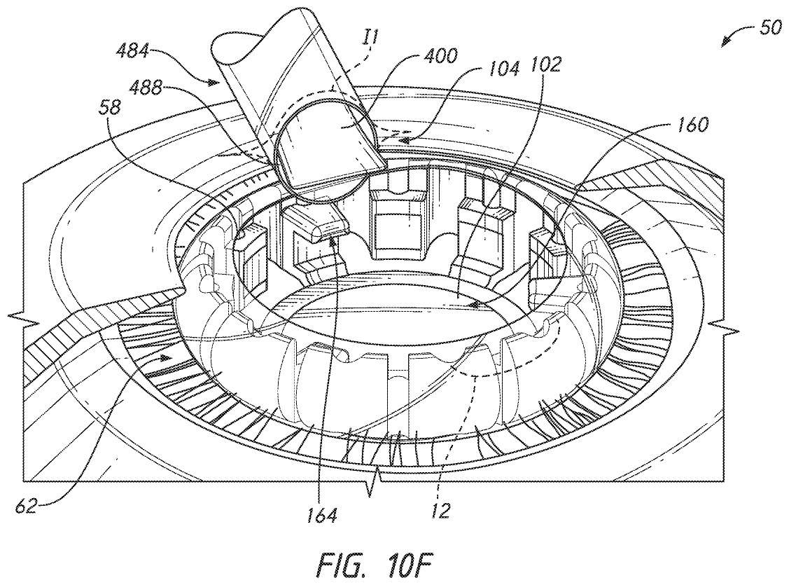

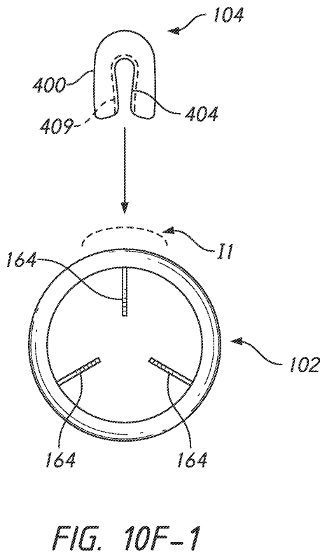

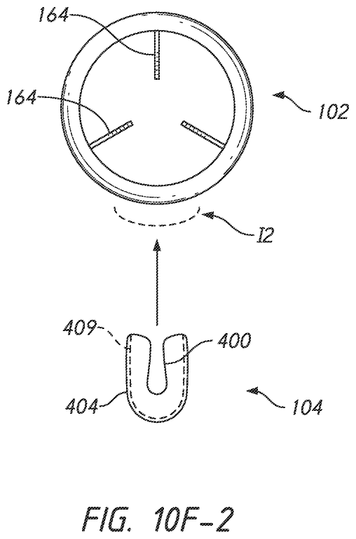

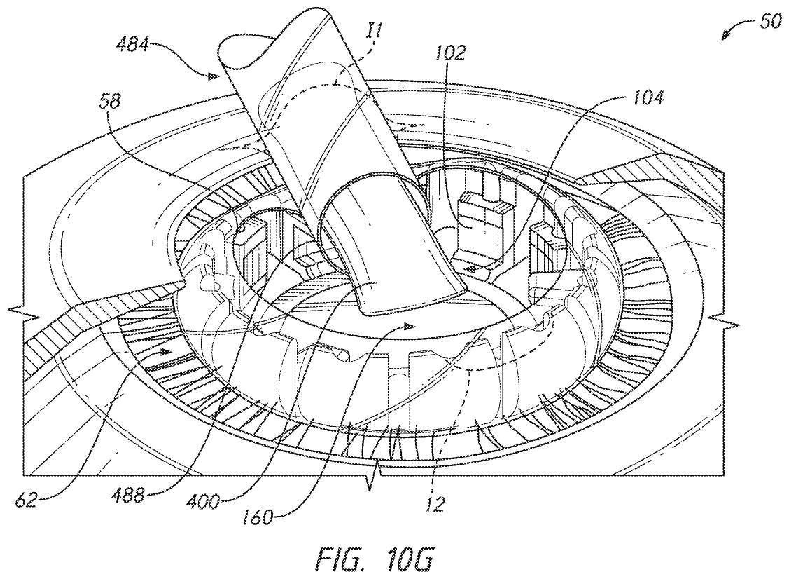

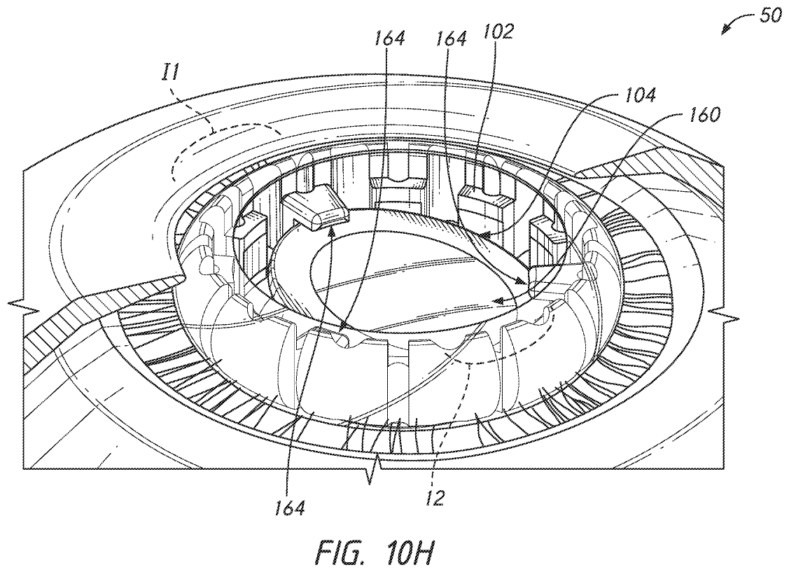

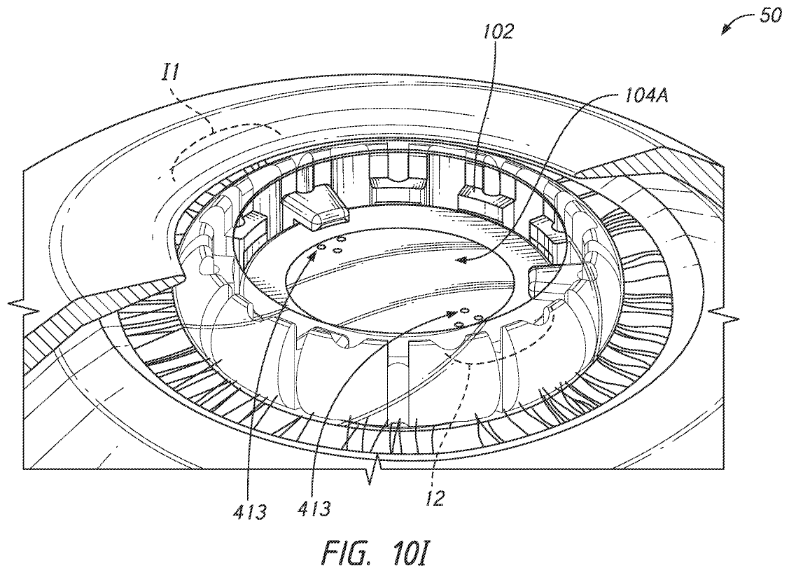

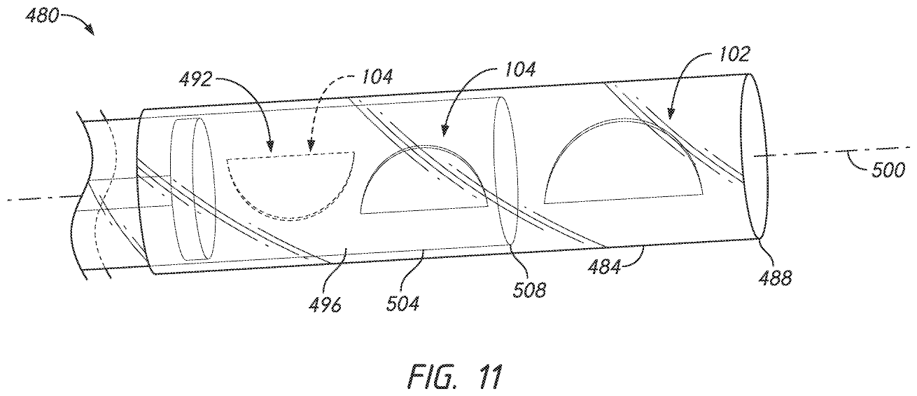

FIGS. 2-2D show variants of the accommodating IOL device 100 shown in FIGS. 1 and 10C-H assembled separate from the eye 50. The accommodating IOL device 100 includes a base member 102 and a power changing lens 104. The power changing lens 104 is separate from the base member 102 such that the base member 102 and the power changing lens 104 can be delivered separately, e.g., sequentially. The variants illustrated by FIGS. 2-9 can provide a lower position for the power changing lens 104 than those of FIGS. 1 and 10C-H. The discussion in FIGS. 1 and 10C-H also apply to the variants of FIGS. 2-9. The systems of FIG. 11 apply to all variants herein. The base member 102 can be delivered before the power changing lens 104. The power changing lens 104 can be subsequently delivered into the base member 102 and can be unfolded within the base member 102 when the base member 102 is in the capsular bag 62 of an eye 50. This sequential delivery allows the base member 102 and the power changing lens 104 to have more complex structure, providing premium function and yet still be deliverable through a small incision.

The base member 102 can include a base lens 120 and a haptic 124. The base lens 120, if present, provides some of the focusing power of the accommodating IOL device 100. The haptic 124 extends into the equatorial region 74 of the capsular bag 62 and establishes a mounting position for the power changing lens 104. In one embodiment the base lens 120 and the haptic 124 cooperate to maintain the capsular bag 62 in an expanded state, similar to the shape and size of the crystalline lens 54 prior to the capsulotomy. As such, the base member 102 is large compared to traditional non-premium IOLs which are designed for delivery through a small incision.

The power changing lens 104 includes multiple optical components, e.g., a membrane at a first side 400 and a lens at a second side 404. An optical fluid can be disposed between the first side 400 and the second side 404 The power changing lens 104 includes a peripheral portion 408 that together with the optical components at the first side 400 and second side 404 contain the optical fluid. The optical fluid has a number of advantages, including transferring compressive forces from the peripheral portion 408 of the power changing lens 104 to the deformable optical surfaces in a controlled manner to provide optically acceptable surfaces across the range of accommodation. The structure of the power changing lens 104 is much more complex than a traditional non-premium IOL, to provide premium function.

Separating the base member 102 and the power changing lens 104 prior to insertion into the eye enables a smaller incision size than were all the optical components inserted simultaneously as unit. The base member 102 and the power changing lens 104 can be compressed to a greater extent when separate than when they are combined into an assembly. Additionally, forming the base member 102 separate from the power changing lens 104 enables the base member 102 to be used in other IOL devices that may not necessarily be accommodating.

FIG. 2A shows that the accommodating IOL device 100 can have one or a plurality of open channels 108 when assembled. The open channels 108 extend in an anterior and posterior direction between a posterior side of the base member 102 and an anterior side of the power changing lens 104. There are twelve open channels 108 in the illustrated embodiment. There can be more or fewer open channels 108, e.g., at least two, at least four, at least six, at least eight, or at least ten open channels 108. Preferably there are an even number of channels arranged symmetrically relative to an optical axis A of the IOL device 100. Each of the open channels 108 is defined in part by an inner periphery 144 of the base member 102 and by the outer periphery or outer peripheral portion 408 of the power changing lens 104. The open channels 108 can each be disposed between adjacent compression arms 180 of the base member 102, which are discussed further below. The open channels 108 allow fluid to circulate in the capsular bag 62, e.g., to flow between anterior and posterior sides of the device 100 and to flow from areas outside the optical zone of the accommodating IOL device 100 to within the optical zone. This fluid flow can reduce the tendency of the accommodating IOL device 100 to create pressurized zones between the capsular bag 62 and the surfaces of the components of the device 100.

FIG. 2C shows the accommodating IOL device 100 can have one or a plurality of open channels 109 when assembled that can also provide for flow from outside the accommodating IOL device 100 into a space 112 disposed between the base member 102 and the power changing lens 104. FIG. 2C shows fluid flow 110 that can be provided through the open channels 109. As discussed further below, the channels 109 are configured to permit fluid flow but to restrict migration of cells into the space 112. The channels 108 are provided in a posterior-anterior direction to provide or enhance flow 110 between the anterior and posterior sides of the accommodating IOL device 100 as shown in FIGS. 2 and 2A. The plurality of open channels 109 can allow fluid to flow from outside the accommodating IOL device 100 to a location between the base lens 120, if present, and the second side 404 of the power changing lens 104. By opening the space 112 to flow of fluid from outside the accommodating IOL device 100 to a location between the base lens 120 and the second side 404, the application of force in a direction transverse to the optical axis OA rather than along the optical axis OA is the primary cause of power change. The transfer of forces from the equatorial region 74 through the base member 102 to the power changing lens 104 can be uniquely configured to cause accommodation upon uniformly dispersed radial and circumferential compression.

FIGS. 2B, 3A, 10D-10F and 10G-10H show that the base member 102 has a cavity 160 configured to receive and retain the power changing lens 104. The cavity 160 is defined between the base lens 120 and an opening 136 at an opposite end of the base member 102 and is surrounded by a haptic 124 disposed at a periphery of the accommodating IOL device 100. More specifically, the base lens 120 includes an anterior surface 122 that faces the cavity 160. The anterior surface 122 partly bounds the cavity 160. The base lens 120 also has a posterior surface 123 that faces toward and may contact an anterior side of the posterior portion of the capsular bag 62. In some embodiments, a curvature of the anterior surface 122 can be lesser than a curvature of posterior surface 123. For example, the curvature of the anterior surface 122 can be less than or equal to about 15 mm⁻¹. The curvature of the anterior surface 122 can be greater than 0 and less than or equal to about 12 mm⁻¹, greater than 0 and less than or equal to about 10 mm⁻¹, greater than 0 and less than or equal to about 7 mm⁻¹, greater than 0 and less than or equal to about 5 mm⁻¹, greater than 0 and less than or equal to about 3 mm⁻¹, or any value in a range/sub-range defined by any of these values. As another example, the base lens 120 can be configured as a plano-convex lens having a substantially planar anterior surface 122. In such embodiments, the curvature of the anterior surface 122 can be 0 or substantially equal to 0 (e.g., less than or equal to 0.1 mm⁻¹. Also, the haptic 124 has a first end 128 and a second end 132 opposite the first end 128. The first end 128 is the end of the haptic 124 that is anterior when the base member 102 is placed in the capsular bag 62. The second end 132 is the end of the haptic 124 that is posterior to the first end 128 when the base member 102 is placed in the capsular bag 62. The cavity 160 is disposed between a first edge 152 and a second edge of the haptic 124. As discussed further below, the haptic 124 has anterior and posterior zones disposed about the cavity 160 that are separately configured for retention and compression of the power changing lens 104 and for enhancing overall compressibility of the base member 102.

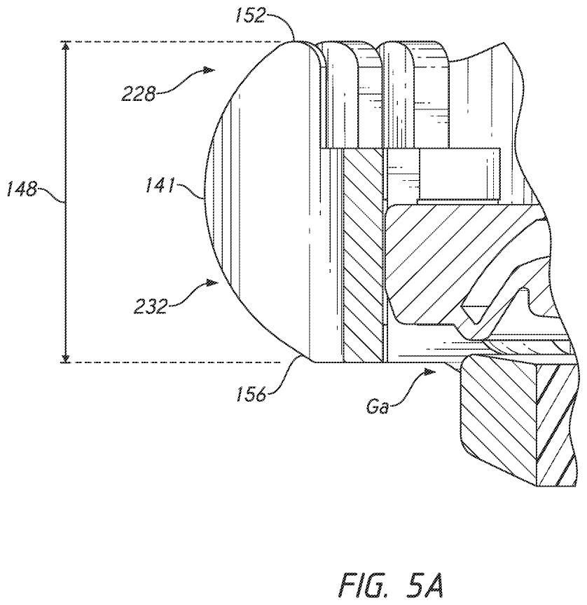

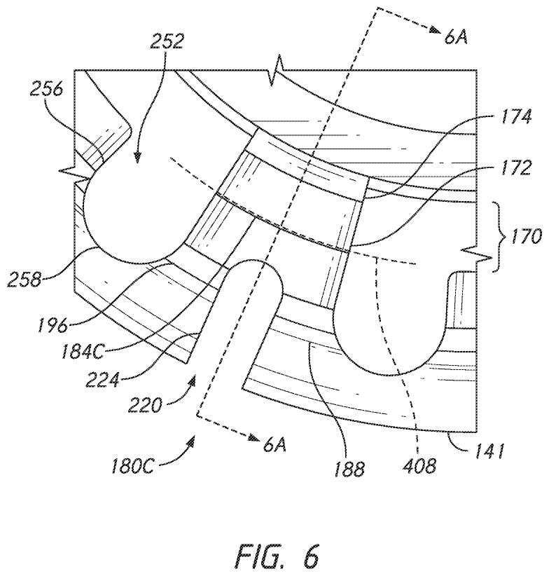

FIG. 2 shows the power changing lens 104 is inset in the cavity 160. As discussed herein, this state can be achieved in the eye in order to reduce or minimize incision size. A first side 400 of the power changing lens 104 is posterior to an opening 136 of the haptic 124 formed at the first end 128 thereof. The configuration of the haptic 124 of the base member 102 assures that the posterior side of the anterior portion of the capsular bag 62 remains spaced away from the posterior portion of the capsular bag 62. The spacing of the two layers of the capsular bag 62 from each other reduces, minimizes, or eliminates fibrosis between these structures which would limit or reduce the potential for accommodative amplitude. A height 148 of the haptic 124 (see FIG. 5A) between a first edge and a second edge of it outer periphery is configured to retain the capsular bag 62 in an open configuration. For example, the height 148 of the haptic 124 can be greater than or equal to about 2 mm. In various embodiments, the height 148 of the haptic 124 can be greater than or equal to about 2.0 mm and less than or equal to about 3.5 mm, greater than or equal to about 2.2 mm and less than or equal to about 3.3 mm, greater than or equal to about 2.5 mm and less than or equal to about 3.0 mm, or any height in a range/sub-range defined by any of these values. The height 148 and profile of the outer periphery 140 are configured to keep anterior portions of the capsular bag 62 anterior of posterior portions of the capsular bag 62. This can reduce, eliminate or minimize fibrosis or “shrink-wrapping” of the capsular bag 62. The height 148 and profile of the outer periphery 140 are configured to keep anterior portions of the capsular bag 62 anterior of the power changing lens 104. This can prevent the capsular bag 62 from interfering with the accommodating performance of the accommodating IOL device 100, as discussed further below. In various embodiments, the haptic 124 can comprise an opaque dye (e.g., dark blue dye, indigo dye, violet dye) to increase the visibility of the haptic during implantation of the power changing lens.

The distance from the opening 136 to the first side 400 of the power changing lens 104 and the configuration of the first end 128 of the haptic 124 provide that the anterior portion of the capsular bag 62 remains spaced away from the power changing lens 104. If the anterior portion of the capsular bag 62 were in contact with the first side 400, the accommodating effect of the power changing lens 104 would be reduced. In various embodiments, the distance from the opening 136 to the first side 400 of the power changing lens 104 can be greater than or equal to about 0.6 mm and less than or equal to about 0.75 mm. In various embodiments, the distance from the opening 136 to the first side 400 of the power changing lens 104 can be about 0.01% of the axial height 148 of the haptic 124 to about 37% of the axial height 148 of the haptic 124. Positioning the power changing lens 104 at a distance of about 0.01% of the axial height 148 of the haptic 124 to about 37% of the axial height 148 of the haptic 124, from the opening 136 can advantageously reduce the risk of retinal detachment and PCO as a result of filling the capsular bag. The base member 102 alone and in combination with various second lenses disclosed herein can have a stable effective lens placement (ELP) and/or reduced post-implantation tilt or rotation issues as a result of filling or maintaining the volume of the natural capsular bag. Filling or maintaining the volume of the natural capsular bag can also result in stable refraction after implantation and/or reduced vitreo-retinal tension. Without ascribing to a particular theory, it is believed that by substantially maintaining the volume of the natural capsular bag the vitreous is prevented from shifting anteriorly. Positioning the power changing lens 104 at a distance of about 0.01% of the axial height 148 of the haptic 124 to about 37% of the axial height 148 of the haptic 124, from the opening 136 can advantageously reduce inflammation after surgery.

The accommodating IOL device 100 includes a lens retention portion 164 configured to maintain the power changing lens 104 in an inset position within the cavity 160. The lens retention portion 164 is spaced away from the opening 136 into the cavity 160 of the haptic 124. The lens retention portion 164 can include a plurality of members, as discussed in greater detail below in connection with several figures.

Having discussed the accommodating IOL device 100 overall, further details of specific features will be discussed in greater depth.

### C. Base Member Configurations

The general structure of the base member 102 is discussed above. FIGS. 2B, 3A-3C and 5-7A illustrate various additional advantageous aspects of the base member 102.

1. Base Lens and Haptic Interface

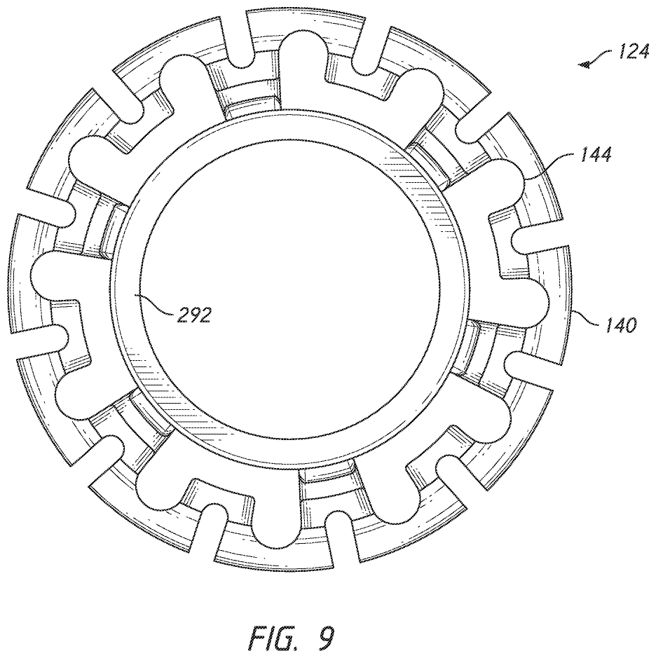

FIGS. 2B and 9 show that the base lens 120 and the haptic 124 can be formed separately and then assembled to form base member 102. In other embodiments the base member 102 is a single molded component with a monolithic structure. The haptic 124 can include the outer periphery 140 configured to contact the equatorial region 74 of the capsular bag 62 and the inner periphery 144 disposed inward of the outer periphery 140 as discussed above. FIG. 9 shows that the haptic 124 also can include a lens interface portion, in one example a ring 292, disposed radially inwardly of the inner periphery 144. The ring 292 can be disposed posteriorly of equator contact segments 141 of the outer periphery 140. The ring 292 can be disposed posteriorly of the second end 132 of the haptic 124. The ring 292 can be disposed posteriorly of the second edge 156 of the haptic 124. The position of the ring 292 relative to the equator contact segments 141 of the outer periphery 140 can be selected to place a posterior aspect of the base member 102 in direct contact with the anterior side of the posterior portion of the capsular bag 62 when the base member 102 is placed in the capsular bag 62. The distance from the ring 292 or from the base lens 120 along the optical axis OA coupled therewith can be known and controlled and can be a factor in selection of the power changing lens 104 or of another non-accommodating lens, as discussed below.

FIG. 2B shows that the base lens 120 can be coupled with the haptic 124 at the ring 292 (or other lens interface portion). The base lens 120 can have a haptic interface surface 320, which is one example of a haptic interface portion or a peripheral haptic interface portion, and the lens interface surface or portion 332 can have a ring 292. In the illustrated embodiment the lens interface surface or portion 332 includes an annular area disposed about the inner periphery of the ring 292. The lens interface surface or portion 332 can include a posterior surface of the ring 292. In the illustrated embodiment the haptic interface surface 320 of the base lens 120 can include an annular skirt 321 disposed about the periphery of the base lens 120. The haptic interface surface 320 can be a complete annulus in one embodiment. In other embodiments the haptic interface surface 320 can include a plurality of spaced apart members that are disposed about the circumference of the base lens 120. The base lens 120 can be coupled with the haptic 124 at the surfaces or portions 320, 332 by any suitable means including using adhesives, welding or by interlocking connectors such as interference fit posts and recesses or features that can be snapped together, eliminating adhesives and stress concentrations or materials transformations associated with welding.

Stated differently, the base member 102 of an intraocular lens 100 can be assembled using the method described below, which includes coupling and securing the base member haptic 124 and the base lens 120. The base member haptic 124 can include the lens interface portion 332 at the second end 132 that is opposite the first open end 128. The lens interface portion 332 can include ring 292. The base lens 120 can have a central optical portion 323 and a haptic interface portion 320. The base lens 120 can have a periphery 325, which can be circular or can be a cylindrical surface of the central optical portion 323 that faces away from the optical axis thereof, and that is sized to be inserted into the ring 292 of lens interface portion 332. The haptic interface portion 320 can have an annular skirt 321.

The haptic interface portion 320 of the base lens 102 can be coupled with the lens interface portion 332 of the base member haptic 124. The cylindrical or circular periphery 325 of the base lens 120 can be inserted into the ring 292 of the lens interface portion 332 of the base member haptic 124 such that an anterior surface of the annular skirt 321 is coupled to a posterior surface 335 of the ring 292. In some aspects, the lens interface portion 332 can include an opaque structure (e.g., a blue colored structure) and the base lens 120 can include an optically transmissive structure such that coupling the haptic interface portion 320 with the lens interface portion 332 includes transitioning from optically transmissive to optically opaque at an interface or boundary between the base lens 120 and the base member haptic 124.

In some aspects, the haptic interface portion 320 includes an annular member, e.g., the skirt 321, disposed radially outward of the central optical portion 323 and the lens interface portion 332 includes an annular structure, e.g., the ring 292, disposed at the second end 132 of the base member haptic 124 such that coupling the haptic interface portion 320 to the lens interface portion 332 includes placing an anterior side of the annular member, e.g., the skirt 321, against a posterior side 335 of the annular structure, e.g., the ring 292. In some aspects, the haptic interface portion 320 includes a periphery 325 and the lens interface portion 332 includes an optical axis facing surface 333 facing an optical axis OA of the base lens 120 such that coupling the haptic interface portion 320 to the lens interface portion 332 includes advancing the periphery 325 of the central optical portion 323 along the optical facing surface 333 of the base member haptic 124.

In some aspects, the haptic interface portion 320 includes a first transverse surface, e.g., an anterior-facing surface of the skirt 321, disposed transverse to an optical axis OA of the base lens 120 and a first annular surface, e.g. the periphery 325, disposed about an optical axis OA. The lens interface portion 332 can include a second transverse surface 335 (e.g., the posterior face of the ring 292) and a second annular surface, e.g., the optical facing surface 333 (e.g., the portion facing toward the center of the space in which the base lens 120 is mounted). Coupling the haptic interface portion 320 of the base lens 120 with the lens interface portion 332 of the base member haptic 124 can include disposing the first annular surface 325 at least partially within the second annular surface 333, and disposing the first transverse surface 321 adjacent to the second transverse surface 335.

The base lens 102 can be secured to the base member haptic 124 at the lens interface portion 332 and the haptic interface portion 320. Securing the lens interface portion 332 and the haptic interface portion 320 can include applying an adhesive between an anterior surface of the annular skirt 321 and a posterior surface 335 of the ring 292. Securing the lens interface portion 332 and the haptic interface portion 320 can include applying an adhesive between an inward surface 333 of the ring 292 and an outward surface 325 of the base lens 120. The adhesive used to secure the lens interface portion 332 with the haptic interface portion 320 and the annular skirt 321 with the ring 292 can be the same material used to form the base lens 102, the haptic 124 and/or other components of the intraocular lens 100, which can include the materials described herein. The adhesive can be applied where the circular periphery 325 and the annular skirt 321 meet, which can result in the formation of a trough. The trough can include an area disposed around the location where the skirt 321 and the periphery 325 meet. The anterior surface of the skirt 321 can be inclined such that the free end thereof is at a higher elevation than the end joined to the periphery 325. This construction helps contain the adhesive during assembly, such that the adhesive is maintained away from the optical surfaces of the base lens 120. The base member haptic 124 can be made of a different material than the base lens 120, but nonetheless, the adhesive used to secure the base member haptic 124 and the base lens 102 can be capable of joining or adhering the two different materials.

By forming the base lens 120 separate from the haptic 124, the base member 102 can benefit from using materials that are adapted for the particular purpose. The base lens 120 can be formed from a material with: high optical quality, high compressibility, low coefficient of friction, beneficial tissue engagement properties for impeding posterior capsule opacification, or with any combination of these material properties. In one embodiment the base lens 120 is formed of silicone, but other materials that could be used include acrylic (e.g., hydrophobic and hydrophilic acrylics). Suitable silicone materials are biocompatible for the haptic 124, including medical grade silicones, where preferably the cured material contains a low, negligible, or medically insignificant volume of compounds extractable by water, saline, or ocular fluids at about 37° C. Certain suitable silicone materials have a Young's modulus when cured of less than 100 psi (about 7×10⁵ Pa), or even less than 50 psi (about 3.5×10⁵ Pa), including 5-50 psi (about 3.5×10⁴-3.5×10⁵ Pa), 10-40 psi (about 7×10⁴-3×10⁵ Pa), and 10-35 psi (about 7×10⁴-205×10⁵ Pa). Examples of suitable silicone materials include, but are not limited to, MED 4805, MED4810, MED4820, MED4830, MED5820, and MED5830 from NuSil®. For the optic examples of suitable silicone materials include, but not limited to, MED 6215, MED6210, MED6219, MED 6233 and MED6820. Suitable optic materials may also include a UV chromophore or UV absorbing group that may be blended with or bonded to a silicone component. In some such materials the UV chromophore or UV absorbing group is substantially non-extractable from the cured lens material by water, saline or ocular fluids at about 37° C. Embodiments of the base lens 120 comprising acrylic can be partially manufactured using molding methods and partially machined. The haptic 124 can be made of a material that is the same as or different from the material of the base lens 120. The haptic 124 can be made of a material that is selected to be selectively stiff or incompressible. As discussed further below, the haptic 124 includes compression arms that preferably transfer a high percentage of force from a radially outward position to a radially inward position to produce a large amount of accommodation in the power changing lens 104 for a unit of ocular force. The material for the haptic 124 can also take into consideration a preference for circumferential compression, low friction coefficient, maintaining bulk properties over a large number of cycles, and other properties. One material suitable for the haptic 124 is silicone, including, but not limited to, the silicone materials listed above for the base lens, but other materials could be used.

In some variations discussed further below the base lens 120 is omitted. The base member 102 can include the ring 292 which can directly contact an annular area of the capsular bag disposed about the optical axis OA. The ring 292 can be extended further posteriorly to provide the same distance to the equator contact segments 141 or the distance can be varied and taken into account when the overall optical design of the power changing lens 104 is selected.

2. Lens Positioning Surfaces

The haptic 124 is configured to set the position of a lens disposed in the cavity 160. The haptic 124 can be configured to set one more of the anterior-posterior location of one or both of the first side 400 and the second side 404 of the power changing lens 104. The haptic 124 can be configured to set the orientation of one or both of the first side 400 and the second side 404 of the power changing lens 104 relative to the optical axis OA of the accommodating IOL device 100.

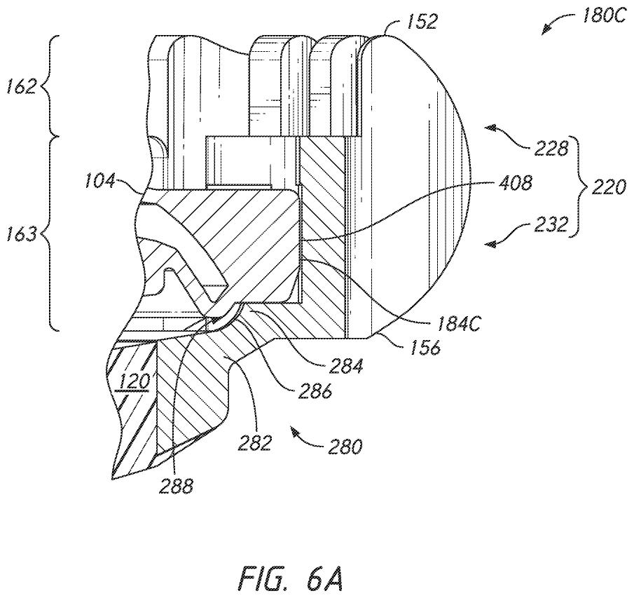

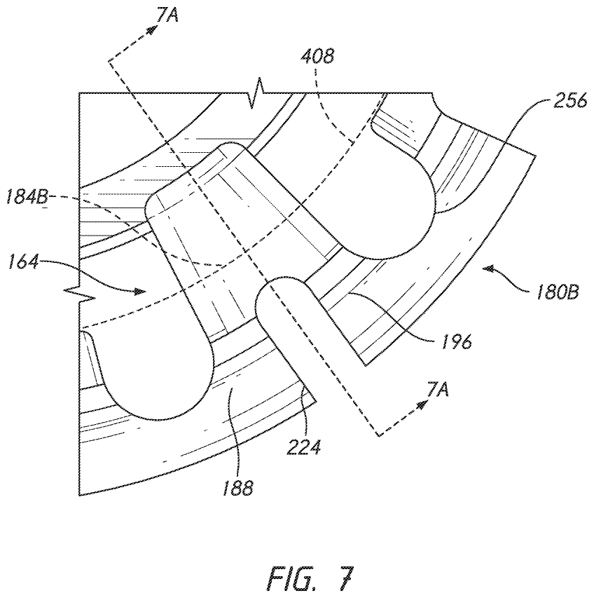

The haptic 124 can have a surface or a plurality of surfaces that mate with the power changing lens 104 to set the position of the power changing lens 104 along the optical axis OA of the accommodating IOL device 100. FIG. 2D shows that the second side 404 of the power changing lens 104 is placed into the cavity 160 and a portion of the peripheral portion 408 on the second side 404 of the power changing lens 104 can come to rest on a plurality of support surfaces 170. The support surfaces 170 can extend radially inward from the posterior end of compression arms 180. Each support surface 170 can have an outer end 172 coupled with a posterior end of a corresponding compression arm 180 and an inner end 174 disposed radially inwardly of the outer end 172 (see FIGS. 6 and 6A).

The circumferential extent of the support surfaces 170 can be the same at each of a plurality of spaced apart locations. The circumferential extent can extend over an arc of approximately 25 degrees, over an arc of approximately 20, over approximately an arc of 15 degrees, over an arc of approximately 10 degrees, or over an arc in a range of approximately 10-30 degrees, or over an arc in a range of approximately 15-20 degrees. The radial extent of the support surfaces 170 can be approximately 2-20% of the diameter of the second side 404 of the power changing lens 104. In other embodiments the radial extent of the support surfaces 170 can be approximately 4-15%, 6-10%, or about 8% of the diameter of the second side 404 of the power changing lens 104.

Preferably at least three of the support surfaces 170 are coplanar with each other. Preferably at least three of the support surfaces 170 are aligned in a common plane that is substantially transverse to, e.g., within about 2-5 degrees of perpendicular to, the optical axis OA of the accommodating IOL device 100. In one embodiment three or more, e.g., all, of the support surfaces 170 are aligned in a plane perpendicular to the optical axis OA. In some cases, the support surfaces 170 are configured to contact the second side 404 of the power changing lens 104 and when in such contact to cause the optical axis of the power changing lens 104 to be less than 25 degrees offset form the optical axis of the base lens 120. The support surfaces 170 can be configured to contact the second side 404 of the power changing lens 104 and when in such contact to cause the optical axis of the power changing lens 104 to be less than 15, less than 10, less than 5 or less than 3 degrees offset form the optical axis of the base lens 120. In various embodiments, the edges of the haptic 124 and/or the base lens 120 can be rounded to reduce or mitigate the occurrence of dysphotopsia. For example, one or more edges in the optical path can be configured as rounded edges instead of sharp edges to reduce or mitigate dysphotopsia. As another example, the edges of the lens retention portion 164, the edges of the equator contact segments 141, the edges of one or more support surfaces 170 can be at least partially configured as rounded edges instead of sharp edges to reduce or mitigate dysphotopsia. Without any loss of generality, a plurality of the edges in a circular region of diameter 7 mm around a geometric center of the IOL device 100 can be configured as rounded edges instead of sharp edges to reduce or mitigate dysphotopsia.

Although the base member 102 is illustrated to have six support surfaces 170, there could be fewer or more than six support surfaces 170. In various embodiments there are four, three or two support surfaces 170 against which the power changing lens 104 is placed to position the power changing lens 104 in the base member 102.

3. Haptic With Enhanced Circumferential Compressibility

The base member 102 and in particular the haptic 124 preferably has a high degree of compressibility to enhance insertion into the eye 50 and placement in the capsular bag 62. One or both of the outer periphery 140 and the inner periphery 144 can be configured to enhance the circumferential compressibility of the haptic 124. Although it is useful for the base member 102 to be rigid in a radially direction in select position, enabling the base member 102 to be circumferentially compressed allows the base member 102 to be inserted into the eye through a smaller incision. Also, circumferential flexibility allows small local shifting of zones of the haptic 124 during placement in the eye or during accommodation to enhance radial transmission of compression loads to the inner periphery 144 and the power changing lens 104 coupled therewith.

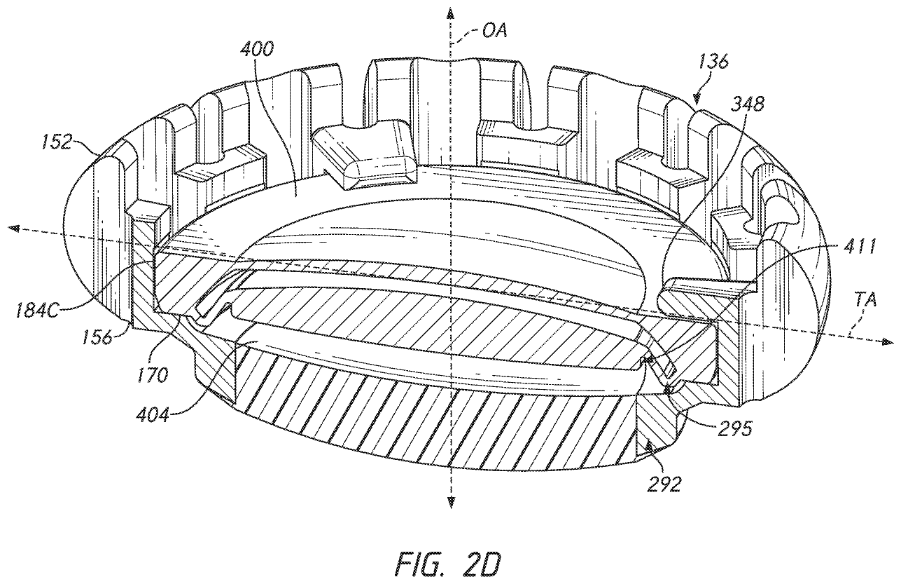

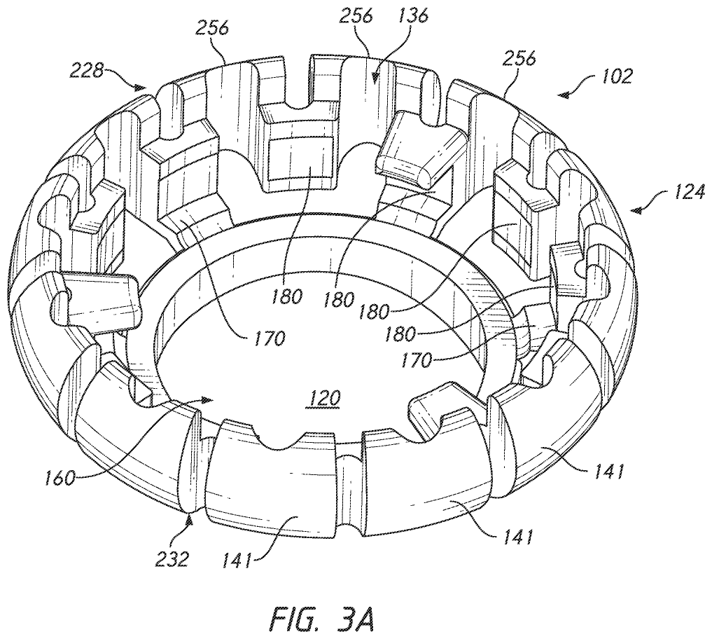

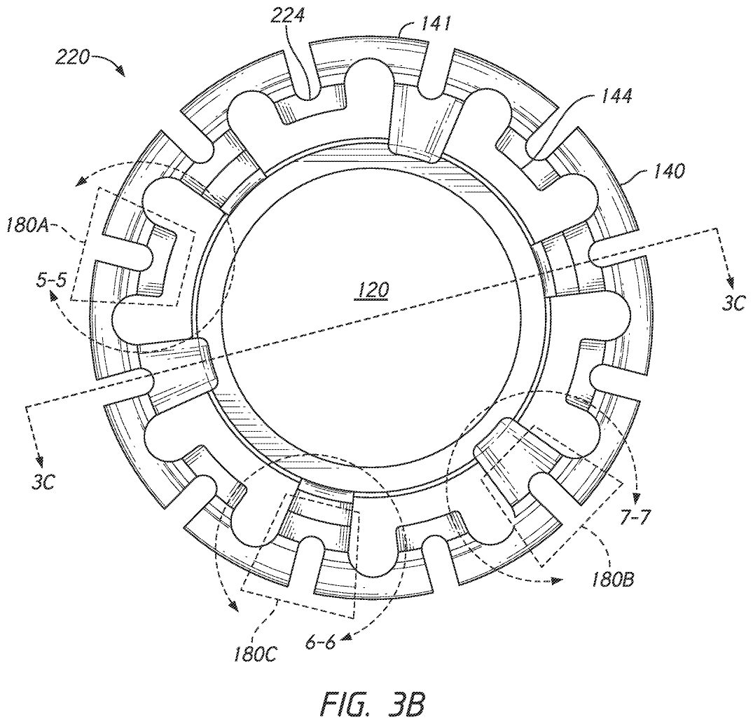

FIG. 3B shows that the outer periphery 140 can include an undulating periphery including a plurality of equator contact segments 141. Each pair of adjacent equator contact segments 141 is separated by an external groove 224. The external grooves 224 alternate between the equator contact segments 141. Each equator contact segments 141 can be disposed between and bounded by an adjacent external groove 224. Each adjacent pair of equator contact segments 141 can be separated by an intervening external groove 224. The presence of the external groove 224 provides spaced apart contact regions with the equatorial region 74 of the capsular bag 62. At each external groove 224 there is no contact with the equatorial region 74 of the capsular bag 62 at that location. The portion of the outer periphery 140 in direct compression transferring contact with the equatorial region 74 of the capsular bag 62 can be greater than or equal to about 50% of the circumference. For example, 50%-100% of the outer periphery 140 in direct compression transferring contact with the equatorial region 74 of the capsular bag 62. As another example, the portion of the outer periphery 140 in direct compression transferring contact with the equatorial region 74 of the capsular bag 62 can be between about 55%-95%, between about 60%-90%, between about 75%-85%, or any value in any range/sub-range defined by these values. The portion of the outer periphery 140 in direct compression transferring contact with the equatorial region 74 of the capsular bag 62 can be greater than or equal to 180 degrees of the 360 degrees of the circumference. For example, portion of the outer periphery 140 in direct compression transferring contact with the equatorial region 74 of the capsular bag 62 can be in the range of 180-350 degrees of the 360 degrees of the circumference. As another example, the entire 360 degrees of the circumference of the outer periphery 140 can be in direct compression transferring contact with the equatorial region 74 of the capsular bag 62.

The inner periphery 144 also has an undulating configuration in one embodiment. A plurality of compression members 180 are spaced apart from each other about the inner periphery 144. FIGS. 2A and 3A show that the contact zones 184 of an array of compression arms 180 are configured to engaged corresponding spaced apart segments of the outer circumference of the peripheral portion 408 of the power changing lens 104. The segments of the outer circumference of the peripheral portion 408 that are engaged by the contact zones 184A are spaced apart by alternating zones of no contact. When the power changing lens 104 is disposed in the base member 102 a substantial minority of the circumference of the peripheral portion 408 of the power changing lens 104 is out of contact with the inner periphery 144 due to the undulating configuration of the inner periphery 144. The compression members 180 apply compressive forces to the peripheral portion 408 at the spaced apart zone of contact as discussed further below. An internal groove 256 can be provided between adjacent pairs of contact zone 184A of adjacent pairs of compression arms 180.

In some embodiments, one or more of the external groove 224 is configure as an external hinge 220. The external hinge 220 can be disposed in a portion of one or more of the compression arms 180. The external hinge 220 provides for flexing at the outer periphery 140 of the haptic 124. The flexing can cause adjacent portions of the compression arms 180 to move circumferentially toward or away from each other either in the process of compressing the base member 102 for implantation or when ocular forces are being applied to the base member 102. The amount of flexibility of the external hinge 220 can be enhanced by extending the external groove 224 farther toward the inner periphery 144. As a radially inward portion 226 of the groove is configured closer to the inner periphery 144 bending and folding of the outer periphery 140 can be enhanced at the same or lower loads.

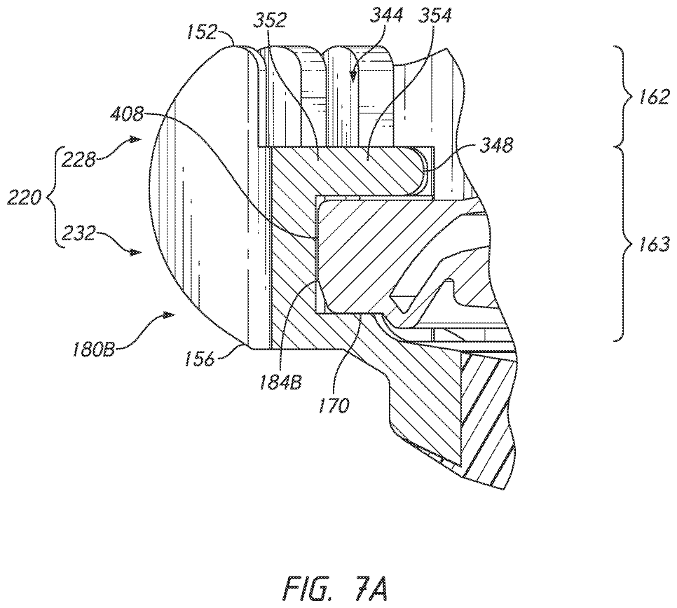

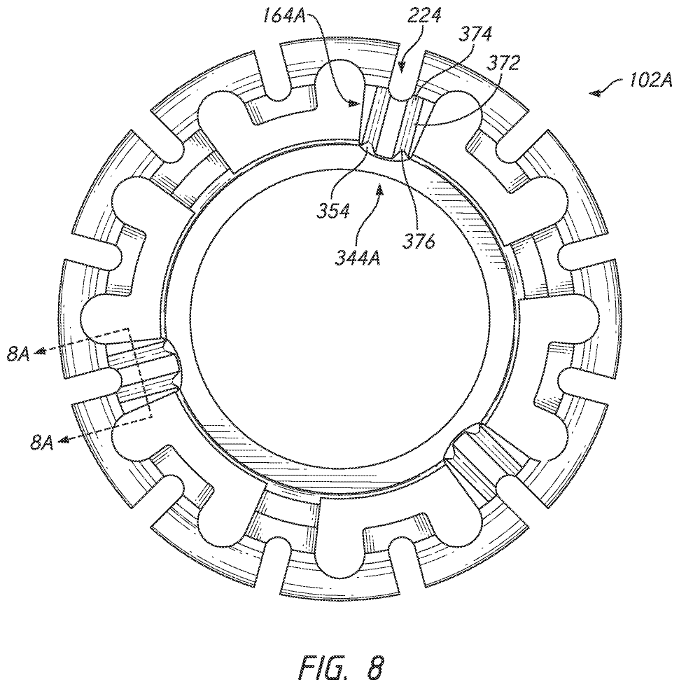

The compressibility of the base member 102 can also be enhanced by enhancing the compressibility of an anterior portion 228 of the external hinge 220 in an anterior segment 162 of the haptic 124 while maintaining or even enhancing the stiffness of a posterior portion 232 of the external hinge 220 in a posterior segment 163 of the haptic 124. FIG. 7A shows that the posterior segment 163 corresponds to the location of the haptic 124 configured to receive and retain the power changing lens 104 (or another premium lens). The anterior segment 162 is disposed between the posterior segment 163 and the first edge 152 of the haptic 124. As can be seen, the anterior portion 228 includes a span of the haptic 124 where circumferentially adjacent portion of the haptic 124 are not connected to each other whereas the posterior portion 232 provides a connection between adjacent segments in the form of the contact zones 184A, 184B, 184C. This configuration allows the haptic 124 in the anterior segment 162 to be more compressible. Also, the external groove 224 can be seen to extend through the entire radial thickness of the haptic 124 from the outer periphery 140 to the inner periphery 144 in the anterior segment 162 of the haptic 124. The external groove 224 can be seen to extend only as far as the portion of the compression arms 180 disposed radially outward of the contact zones 184A, 184B, 184C.

The internal groove 256 can be configured as a portion of an internal hinge 252 in some embodiments. The internal groove 256 extends to a radially outward portion 258. When configured as part of the internal hinge 252, the internal groove 256 extends far enough to provide compressibility of the haptic 124 at the inner periphery 144 at a low force. In some embodiments to greatly increase flexibility of the haptic 124 for circumferential compression, the radially outward portion 258 of the internal groove 256 is radially outward of the radially inward portion 226 of the external groove 224. Less circumferential compressibility is provided if the radially outward portion 258 of the internal groove 256 is radially inward of the radially inward portion 226 of the external groove 224.

The hinges, grooves, and undulating configurations of various embodiments of the haptic 124 enhance circumferential compression without sacrificing transfer of compressive forces from the outer periphery 140 to the inner periphery 144 in the haptic 124 due to the configuration of a plurality of compression arms 180.

4. Array of Arm Providing Power Changing Lens Compression

The accommodating IOL device 100 has a plurality of compression arms 180 configured to convey ocular forces from the equatorial region 74 of the capsular bag 62 to the peripheral portion 408 of the power changing lens 104. The illustrated embodiment shows that the haptic 124 can have twelve compression arms 180 disposed in an array about the cavity 160. The compression arms 180 can all have the same configuration or, as illustrated, can have more than one, e.g., two or three distinct configurations. The base member 102 can include a plurality of sets of compression arms 180. The base member 102 can have a first configuration compression arm 180A that includes a floating contact zone 184A. The base member 102 can have a second configuration compression arm 180B that has a retention contact zone 184B. The base member 102 can have a third configuration compression arm 180C that has a hinged contact zone 184C. Each of these distinct configuration compression members 180 provides distinct function, compression performance, and advantages as discussed below including but not limited to stabilizing the refractive performance of the IOL device 100, minimizing rotation of the IOL device 100 after implantation, improve ease of implantation of the IOL device 100 and/or improve ease of removing the implanted power changing lens 104 to replace with a different power changing lens at a future time.

a. Floating Compression Arms

FIGS. 3A, 3B, 5 and 5A show that the compression arm 180A can be provided at some circumferential positions to contact a peripheral portion 408 of the power changing lens 104. The compression arm 180A is sometimes also referred to as a floating compression arm or a floating compression member because the force transferring portion disposed at the inner periphery 144 is not directly connected to another part of the haptic 124 or to the base lens 120. The power changing lens 104 is omitted from FIG. to simplify the drawing, but the outer circumference of the peripheral portion 408 is shown in dash line. In one embodiment, the compression arm 180A includes a floating contact zone 184A. The floating contact zone 184A is a radially inward portion of the compression arm 180A that comprises a portion of the inner periphery 144 of the haptic 124. The floating contact zone 184A can have a circumferential surface that extends between a first circumferential end 192 and a second circumferential end 200. FIGS. 3C and 5A also show that the floating contact zone 184A has an anterior-posterior extent and thus the floating contact zone 184A can be seen to have a curved but generally rectangular configuration. The anterior end of the floating contact zone 184A is spaced from a first edge 152 of the haptic 124. The contact between the peripheral portion 408 of the power changing lens 104 and the floating contact zone 184A is on a circumferential radially outwardly facing area of the peripheral portion 408. The contact between the peripheral portion 408 and the floating contact zone 184A does not include contact on the first side 400 or the second side 404 of the power changing lens 104. The contact can be over a fraction of the anterior-posterior length of the floating contact zone 184A, e.g., over only 75% of that length, over 65% of that length, over 60% of that length, over 55% of that length, over 50% of that length, or over a length in the range of 40-80% of that length.

The circumferential extent of the floating contact zone 184A can depend on how many contact zones are provided about the inner periphery 144. In one embodiment there are six floating contact zone 184A and each contact zone extends over an arc of approximately 25 degrees, over an arc of approximately 20 an arc of degrees, over approximately an arc of 15 degrees, over an arc of approximately 10 degrees, or over an arc in a range of approximately 10-30 degrees, or over an arc in a range of approximately 15-20 degrees.

FIGS. 3A and 3B show that in some embodiments the floating contact zone 184A are connected to adjacent compression arms 180 in a circumferential direction but are spaced apart from and the base lens 120. The floating contact zones 184A are connected to the adjacent compression arms 180 through relatively flexible connections, e.g., at internal grooves 256 or at internal hinge 252. The floating contact zones 184A are separated from other portions of the base member 102, e.g., from an outer periphery of the base lens 120, by a gap Ga (labeled in FIG. 3B). FIG. 3B shows that the floating contact zone 184A and the gap Ga between floating contact zone 184A and the base lens 120 can be provided in one-half the contact zones (e.g., six of twelve) about the inner periphery 144. The gap Ga can be approximately constant along the circumferential direction of the floating contact zone 184A.

Because the floating contact zones 184A are in contact with the peripheral portion 408 only at the outwardly facing area of the outer circumference of the peripheral portion 408 there is no need to fit the first side 400 of the power changing lens 104 posterior to any aspect of the floating contact zone 184A or the compression arm 180A. This simplifies assembly while still enabling a compression force to be applied at the locations of the compression arm 180A. Also, because the floating contact zones 184A are in contact with only the outwardly facing area of the outer circumference there is no need to seat the second side 404 on an anteriorly facing portion of the compression arm 180A in the peripheral portion 408 of the power changing lens 104. This minimizes any rocking effect that can occur with variability in anterior-posterior position of a support surface configured to facilitate positioning of the power changing lens 104 along the optical axis OA of the accommodating IOL device 100.

In addition to the floating contact zone 184A, the compression arms 180A each include a first radial force member 188 and a second radial force member 196. Each of the force members 188, 196 is configured to transfer a compressive force from the outer periphery 140 of the haptic 124 to the inner periphery 144 of the haptic 124 during accommodation. The first radial force member 188 can be coupled at a first end with an end of a first equator contact segment 141 and at a second end with the first circumferential end 192 of the floating contact zone 184A disposed radially inwardly of the first end of the equator contact segments 141. The second radial force member 196 can be coupled with a first end to an equator contact segments 141 adjacent to the equator contact segments 141 to which the first radial force member 188 is connected. A second end of the second radial force member 196 can be coupled to the second circumferential end 200 of the floating contact zone 184A.

As discussed above, the external groove 224 is provided in the outer periphery 140 of the base member 102. The external groove 224 can be disposed between radially outer portions of the first radial force member 188 and the second radial force member 196. The external groove 224 can extend sufficiently radially inwardly to provide an external hinge in the compression arm 180A. By providing an external hinge in the compression arm 180A, the response of the haptic 124 to circumferential compression of the eye can be maintained while providing enhanced circumferential compression. For example, as ocular forces create compression at the outer periphery 140 of the haptic 124 the facing edges of the equator contact segments 141 at the ends of the first radial force member 188 and the second radial force member 196 may be deflected toward each other (closing the gap around the section plane 5A-5A in FIG. 5). In this configuration the first radial force member 188 and the second radial force member 196 support each other enhancing transfer of radial forces inwardly to the power changing lens 104. Additionally, the internal hinges 252 disposed between adjacent compressions arms 180 can advantageously enable a greater amount of compressive force from the eye to be focused in the radial force member 188, 196. Without being bound by a particular theory, it is believed that this is because the internal hinges 252 bend at lower force and thus more of the total force applied to the haptic 124 is directed radially inwardly along and by the radial force member 188, 196 which together urge the contact zones 184 to the outer circumference of the peripheral portion 408 of the power changing lens 104.

b. Compression Arm With Retention Portion

FIGS. 3B, 7 and 7A show details of a compression arm 180B that can comprise one of a second set of compression arms. The compression arm 180B is similar to the compression arm 180A except as described differently herein. The compression arm 180B includes a retention contact zone 184B that includes a curved but generally rectangular contact zone for engaging an outer circumference of the peripheral portion 408. The compression arm 180B includes a lens retention portion 164 that projects from an anterior end of the retention contact zone 184B. The retention contact zone 184B, the lens retention portion 164 and the support surfaces 170 form a generally C-shaped space of the compression arm 180B in which an arcuate segment of the peripheral portion 408 is received. The compression arm 180B surrounds three sides of the power changing lens 104 at the peripheral portion 408 on the first side 400, the, second side 404, and on an outer circumference disposed between the first side 400 and the second side 404. The support surfaces 170 supports a posterior side segment of the peripheral portion 408, the retention contact zone 184B supports a circumferential segment located on a radially outward side, and a posterior side of the lens retention portion 164 faces and supports an anterior segment of the peripheral portion 408.

An anterior side of the lens retention portion 164 is recessed from the first edge 152 by an amount that allows the power changing lens 104 to be at a low profile position. For example, the power changing lens 104 can be recessed from the first edge 152 by an amount greater than or equal to about 0.6 mm and less than or equal to about 0.75 mm. For example, the power changing lens 104 can be recessed from the first edge 152 by a distance between about 0.01% of the distance from the first edge 152 to the second edge 156 of the haptic 124 and about 37% of the distance from the first edge 152 to the second edge 156 of the haptic 124. The anterior side of the lens retention portion 164 is disposed adjacent to an opening 167 between the first radial force member 188 and the second radial force member 196. The opening 167 extends from the first edge 152 down to a top edge of the retention contact zone 184B. The opening 167 extends by more than 10% of the distance from the first edge 152 to the second edge 156 of the haptic 124. The opening 167 extends by more than 20% of the distance from the first edge 152 to the second edge 156 of the haptic 124. The opening 167 extends by more than 30% of the distance from the first edge 152 to the second edge 156 of the haptic 124. The opening 167 extends from 10% to 40% of the distance from the first edge 152 to the second edge 156 of the haptic 124. The opening 167 provides enhanced flexibility in the anterior segment 162 of the haptic 124.

The compression arm 180B advantageously provides three distinct functions. First the compression arm 180B provides for radial compression of the power changing lens 104 by transferring compression of outer periphery 140 to the inner periphery 144. The force can be transferred from the equator contact segments 141 to the first radial force member 188 and to the second radial force member 196. The force can be transferred through the first radial force member 188 and second radial force member 196 in substantially equal amounts to the retention contact zone 184B. Because the retention contact zone 184B joins the radially inward ends of the radial force member 188, 196 any lack of uniformity can be balanced across the retention contact zone 184B. A further function of the compression arm 180B is axial position control. The support surface 170 at the compression arm 180B provides axial position control to maintain the power changing lens 104 at a selected position. This function prevents the power changing lens 104 from being positioned farther posteriorly than planned and can help avoid poor distance vision in an unaccommodated state. The lens retention portion 164 provides a third function of the compression arm 180B. The lens retention portion 164 prevents the power changing lens 104 from shifting axially along the optical axis OA of the accommodating IOL device 100 after the accommodating IOL device 100 is assembled in the eye 50. Axial shifting could move the power changing lens 104 out of position in the cavity 160. In an extreme case the power changing lens 104 could come out of the base member 102, e.g., anterior of the capsular bag 62. However, even movement partly or completely into the anterior segment 162 of the haptic 124 would degrade both distance vision and accommodation. Distance vision would be degraded because the unaccommodated position could be configured for the position in the posterior segment 163 of the haptic 124. Accommodation would be degraded because the inner periphery of the haptic 124 in the anterior segment 162 has a larger inner diameter than the inner diameter defined by the retention contact zone 184B and the opposing contact zone, e.g., the hinged contact zone 184C. This larger inner diameter of the inner periphery 144 is larger than the outer diameter of the power changing lens 104, e.g., of the peripheral portion 408 of the power changing lens 104. Accordingly the inner periphery 144 may not even be in contact with the outer circumference of the power changing lens 104 when the accommodating IOL device 100 is in the unaccommodated state.

FIG. 7A shows that the distance in the anterior-posterior direction between the support surfaces 170 and the lens retention portion 164 is close to the thickness of the power changing lens 104, between the first side 400 and the second side 404.

c. Compression Arm with Device Support Surface

FIGS. 3B, 6 and 6A show details of the compression arm 180C which can be one of another set of compression arms. The compression arm 180C is similar to the compression arm 180B but does not include the lens retention portion 164. The power changing lens 104 is exposed on the first side 400 as shown in FIG. 2D.

The compression arm 180C includes a hinged contact zone 184C disposed at a radially inward end of the first radial force member 188 and the second radial force member 196. The radial force members convey ocular forces from adjacent equator contact segments 141 to the hinged contact zone 184C. The hinged contact zone 184C includes a curved surface that extends between the radial force members 188, 196. The anterior edge of the hinged contact zone 184C is open or exposed radially inwardly and anteriorly thereof. The posterior edge of the hinged contact zone 184C is coupled with the outer end 172 of the support surface 170. The compression arm 180C provides engagement with the power changing lens 104 on two surfaces. The second side 404 in the peripheral portion 408 of the power changing lens 104 faces and is supported by the support surface 170. The support surface 170 of the compression arm 180C provides axial positioning of the power changing lens 104 along the optical axis OA. A second surface of engagement is provided at the hinged contact zone 184C, which contacts the outer circumference of the peripheral portion 408 of the power changing lens 104.

An advantage of the compression arm 180C is that axial positioning can be provided to the power changing lens 104 and compression force can be applied to the outer circumference at the peripheral portion 408. The first radial force member 188 and the second radial force member 196 are able to move circumferentially relative to each other due to the external groove 224 which can be configured to provide an external hinge 220. A further advantage is that these functions can be provided with having to position the first side 400 posterior to a retention portion or tab when assembling the power changing lens 104 to the base member 102 in the eye 50.

5. Radial Hinge Coupling of Arms and Base Lens

As discussed further below, the base member 102 is configured to fill the capsular bag 62 in part to restore the capsular bag 62 to a volume similar to the volume of the crystalline lens 54 prior to the capsulotomy. There are several benefits to restoring the volume of the capsular bag 62, as discussed above, including reducing, minimizing or preventing fibrosis between anterior and posterior portions of the capsular bag 62, establishing a predictable and stable position for the power changing lens 104 or other secondary ocular device to be placed in the base member 102, and/or engaging the outer periphery 140 of the haptic 124 with the equatorial region 74 of the capsular bag 62. The base member 102 can be configured to consistently position the equator contact segments 141 at the equatorial region 74.

In one embodiment one or a plurality of radial hinges 280 are provided disposed between the ring 292 and the inner periphery 144 of the haptic 124. The radial hinges 280 extend along opposing segments of three diameters of the radial hinge 280, e.g., at the diameter aligned with section plane 3C-3C in FIG. 3B and at two diameters spaced +60 degrees and −60 degrees from the section plane 3C-3C. The radial hinges 280 are sometimes referred to as diametrical hinges herein. FIGS. 3C and 6A show that the radial hinge 280 can extend from a first portion 282 coupled with and assembly including the base lens 120 to a second portion 284 coupled with a portion of the haptic 124. The first portion 282 can be coupled with the ring 292. The second portion 284 can be coupled with the support surface 170 of one of the compression arms 180. FIG. 3C shows that the first portion 282 of one of the radial hinges 280 can be coupled along an arc of the ring 292 and the second portion 284 can be coupled with the compression arm 180B, e.g., along an arc of the outer end 172 of the support surface 170 coupled with the hinged contact zone 184B. FIG. 3C also shows that the first portion 282 of one of the radial hinges 280 can be coupled along an arc of the ring 292 and the second portion 284 can be coupled with the compression arm 180C, e.g., along an arc of the outer end 172 of the support surface 170 coupled with the hinged contact zone 184C.

The radial hinges 280 can be positioned in an array at a plurality of positions about the base member 102. The radial hinges 280 connect the compression arms 180 to the base lens 120. The connection can be directly to the base lens 120 or can be indirectly, e.g., at the ring 292 as discussed above. FIGS. 3A-3C show that the radial hinge 280 can connect each of the compression arm 180B and the compression arm 180C to the ring 292 and, indirectly, to the base lens 120. The radial hinges 280 are disposed at alternating arms of the plurality of compression arms 180. In the base member 102 there are six radial hinges 280. In the base member 102 three of the radial hinges 280 are disposed at the same angular position as lens retention portion 164 and two of the radial hinge 280 are spaced equally apart from two adjacent lens retention portion 164. The two adjacent lens retention portion 164 can be 120 degrees apart. Two radial hinges 280 can be 60 degrees apart from each of two adjacent lens retention portion 164.

The radial hinge 280 provide at least two functions to the base member 102. First, the radial hinge 280 enable a ring shaped body of the haptic 124 that is disposed between the first edge 152 and the second edge 156 to be compressed to the assembly including the ring 292 and the base lens 120 (if present). The compression of these components together enables the base member 102 to be inserted into the eye 50 in a more compact configuration. The radial hinges 280 also provide freer movement of the inner periphery 144 toward the optical axis OA of the accommodating IOL device 100 upon compression of the capsular bag 62. The posterior surface 123 of the base lens 120 can be placed against the inside anterior-facing surface of the posterior segment of the capsular bag 62. The outer periphery 140 can be placed in the equatorial region 74 of the capsular bag 62. Posterior movement of the posterior surface 123 upon compression of the equatorial region 74 is reduced or prevented by the vitreous fluid posterior to the capsular bag 62. The radial hinge 280 then focus movement of the posterior segment 163 of the haptic 124 radially inward toward the optical axis OA upon compression of the equatorial region 74. The radial hinges 280 are configured such that the second portion 284 tilts radially inwardly toward the optical axis OA upon compression of the inner periphery 144 by ocular forces of the equatorial region 74.

Although six radial hinge 280 are shown, the base member 102 could be configured with fewer or more hinges. Also, although the configurations of the radial hinges 280 can have substantially the same configuration, the radial hinges 280 coupled with the compression arm 180B can have more flexibility in view of the enhanced stiffness imparted by the lens retention portion 164.

6. Lens Retention Portions

As discussed above a critical function of the accommodating IOL device 100 is to retain the power changing lens 104 within the cavity 160 of the base member 102. In the accommodating IOL device 100 the power changing lens 104 is delivered separately from the base member 102, e.g., in the same procedure and through the same incision. The base member 102 is configured to provide enhanced compression of the power changing lens 104 by disposing the power changing lens 104 in the posterior segment 163. This is further desired to retain the base member 102 in the posterior segment 163 of the cavity 160.

In the illustrated embodiment the lens retention portion 164 is provided within the cavity 160. The lens retention portion 164 is disposed at the boundary of the posterior segment 163 and the anterior segment 162. The lens retention portion 164 can include a plurality of, e.g., three, tabs that extend toward the center of the cavity 160. The tabs have a bottom surface configured to be in contact with the power changing lens when implanted and an upper surface facing the opening 136 of the haptic 124. The tabs can be disposed such that an axial distance from the anterior most portion of the haptic 124 to the upper surface of the tab is greater than or equal to about 0.6 mm. For example, the axial distance from the anterior most portion of the haptic 124 to the upper surface of the tab can be greater than or equal to about 0.6 mm and less than or equal to about 0.75 mm, greater than or equal to about 0.7 mm and less than or equal to about 0.9 mm, greater than or equal to about 0.8 mm and less than or equal to about 1.0 mm, greater than or equal to about 0.9 mm and less than or equal to about 1.1 mm, greater than or equal to about 1.0 mm and less than or equal to about 1.25 mm, or any value in any range/sub-range defined by any of these values. In various embodiments, the tabs can be positioned at a distance from the edge of the haptic 124, of about 0.01% of the height 148 of the haptic 124 to about 37% of the height 148 of the haptic 124. The tabs and the support surfaces 170 define spaces for surrounding and holding spaced apart arcs of the peripheral portion 408 of the base lens 120. More particularly, the lens retention portion 164 can be configured as a projection 344 having an outer portion 348, and inner portion 352 and an elongate portion 354 disposed therebetween. The inner portion 352 can be coupled with, an extension of or disposed adjacent to an anterior portion of the hinged contact zone 184C of the compression arm 180C. The elongate portion 354 can extend radially, or along a diameter of the base member 102. The elongate portion 354 can include a planar posterior surface that faces and contacts the first side 400 of the power changing lens 104. FIGS. 2A and 2D show that the outer portion 348 extends well inward of the outer circumference of the power changing lens 104. The outer portion 348 can extend to a position radially between the outer circumference of the power changing lens 104 and the outer periphery of a flexible membrane 402 of the power changing lens 104. The outer portion 348 can be located radially outward of an outer circumference of the flexible membrane.

As discussed further below, the peripheral portion 408 of the power changing lens 104 can include an annular segment that is outward of the optical surfaces thereof. The annular segment can extend between an outer circumference of the peripheral portion 408 and a closed cavity 412 of the power changing lens 104. The annular portion can be configured as a solid annulus between the closed cavity 412 and the outer circumference of the peripheral portion 408. The outer portion 348 of the projection 344 can be disposed across the annulus, e.g., at least one-half of the distance from the outer circumference of the peripheral portion 408 to the outer circumference of the membrane 402. The width of the elongate portion 354 between the projection outer portion 348 and the inner portion 352 can extend over an arc of approximately 30 degrees, over an arc of approximately 25 degrees, over approximately an arc of 20 degrees, over an arc of approximately 15 degrees, or over an arc in a range of approximately 15-40 degrees, or over an arc in a range of approximately 20-30 degrees.

The projection 344 preferably is flexible at the outer portion 348 such that the outer circumference of the peripheral portion 408 can be extended under the posterior side of the elongate portion 354. However, the projection 344 is rigid at the inner portion 352 such that compressive forces of the compression arms 180 do not significantly deflect the inner portion 352.

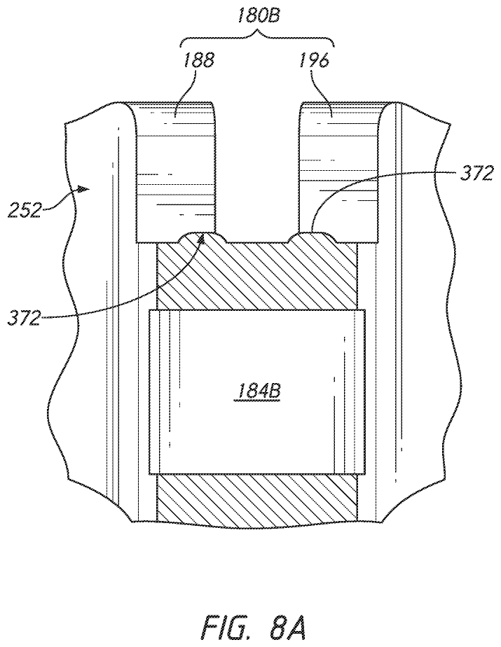

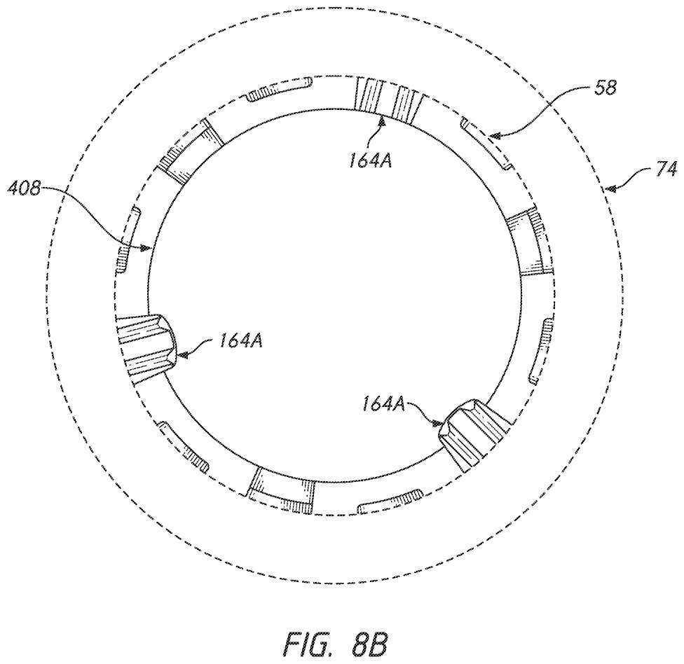

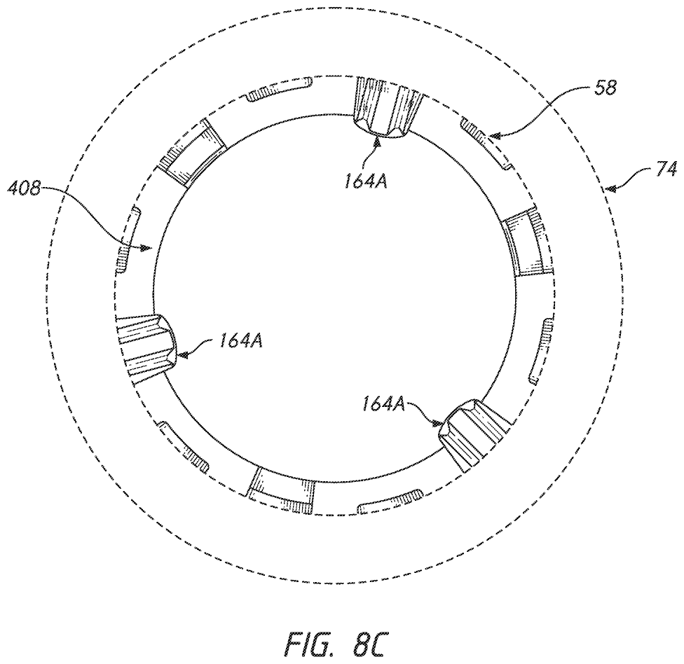

FIG. 8 shows another embodiment of the base member 102A that is similar to the base member 102 in which the lens retention portion 164 is altered. The base member 102A can include any of the structures described above in connection with the base member 102. The base member 102 includes a retention portion 164A configured to enhance visibility of the proper position of the power changing lens 104 within the base member 102. As seen in FIG. 8C, the retention portion 164A can include an end portion that is rounded or curved, which can differ from the more straight end portion of the retention portion 164, as illustrated in FIG. 2A. The lens retention portion 164A includes an elongate portion 354A with a visible guide structure 370. The visible guide structure 370 can include one or a plurality of, e.g., two ridges 372 that are visible when the base member 102A is placed in the eye 50. The ridge 372 includes an outer end 374 and an inner end 376. The outer end 374 can be disposed adjacent to a radially inner end of the external groove 224 in the outer periphery 140. The inner end 376 can be disposed at or adjacent to the radially inner end of the elongate portion 354. FIG. 8A shows a cross-section at section plane 8A-8A in the detail view of FIG. 8.

Any one of the guide structure 370 can be visible to the clinician when the base member 102 has been placed in the eye. If the base lens 120 is axisymmetric, e.g., aspheric or monofocal lacking any cylinder power, the surgeon can simply confirm that all three (or more) of the guide structures 370 on the three (or more) projection 344A are visible as depicted in FIG. 8C. If the base lens 120 has cylinder power the surgeon can confirm that the projection 344A is properly oriented. For example, one of the guide structure 370 can be configured as arrows pointing superiorly when the cylinder power is properly aligned in the eye. If the guide structure 370 of any of the projection 344A is not clearly visible, for example, as shown in FIG. 8B, the surgeon can conclude that the power changing lens 104 is on top of (anterior of) the projection 344A and would be advised to manipulate the projection 344A to place the peripheral portion 408 of the power changing lens 104 posteriorly thereto. The guide structures 372 may be seen to extend radially inward of the outer circumference of the peripheral portion 408 if the projections 344A are posterior to the power changing lens 104. In other techniques, the visible length of the ridges 372 can be measured and if one or more is seen to be shorter in the radial direction than the others, the projection 344A can be concluded to be positioned under the power changing lens 104 and an appropriate adjustment can be made. For example, an instrument can be placed under the projection 344A to lift the projection over (anterior to) the power changing lens 104.

### D. Power Changing and Fixed Power Lenses

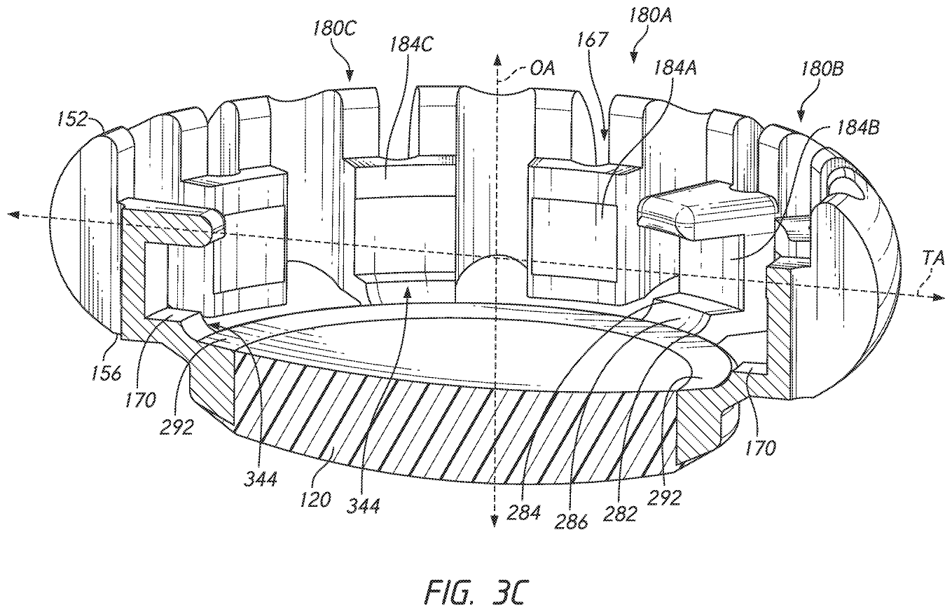

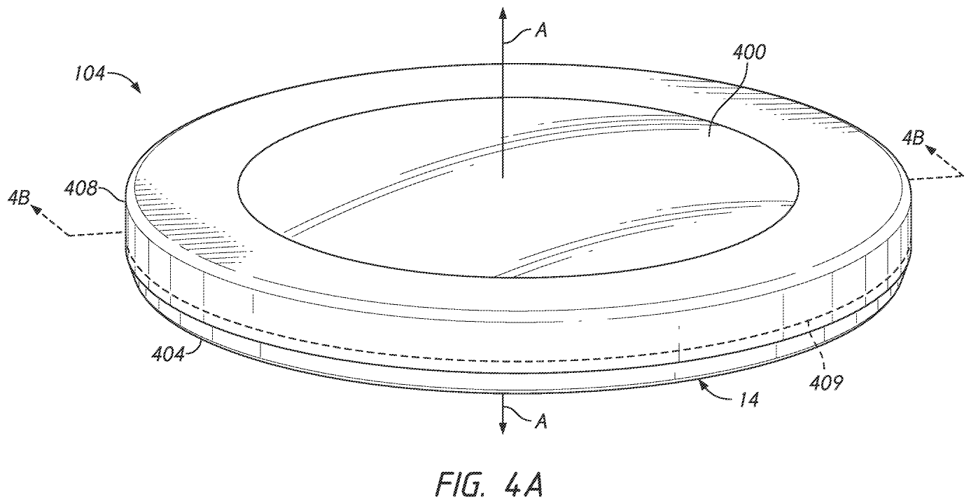

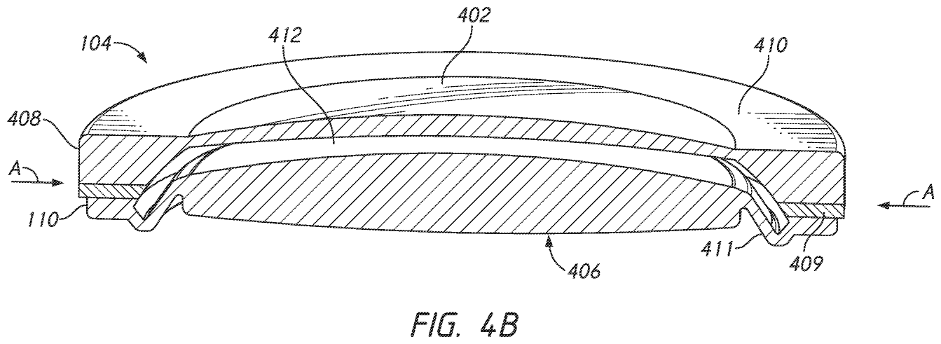

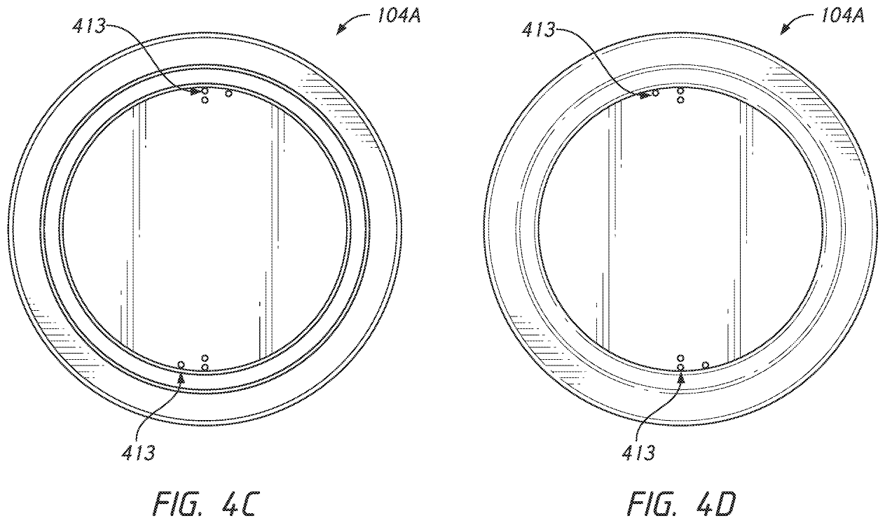

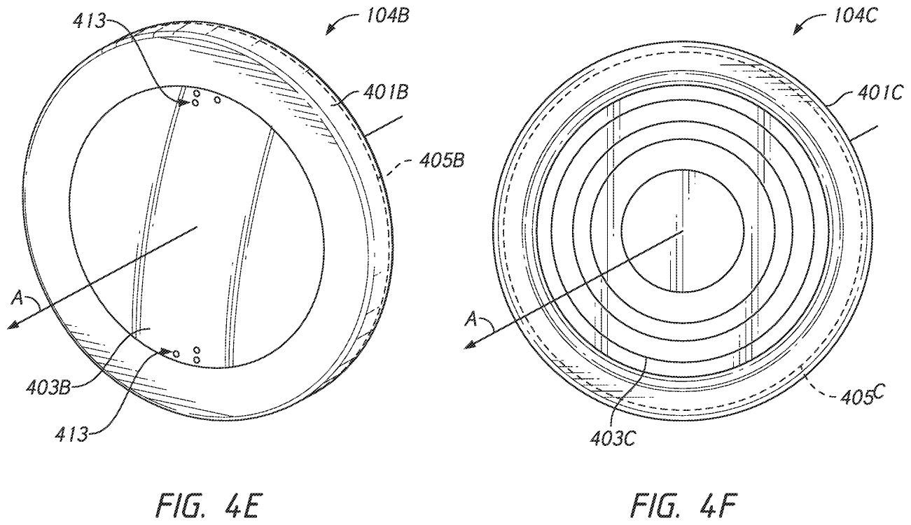

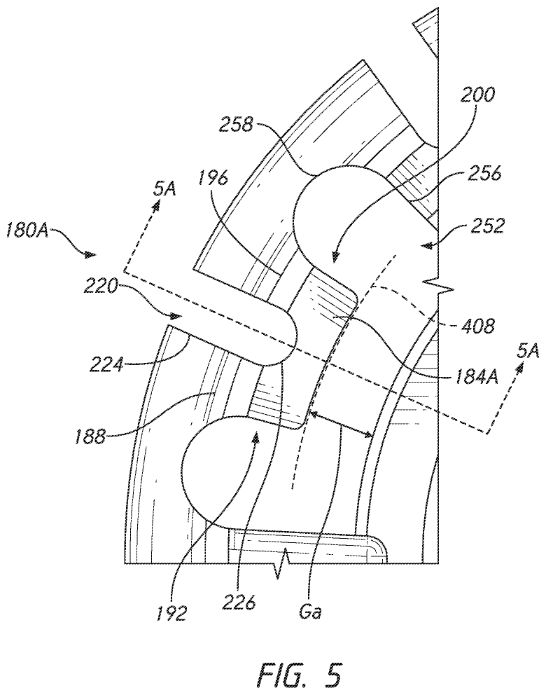

FIGS. 2A-2D and 4A-4D depict various examples of the power changing lens 104 in detail and FIGS. 4E-4F show examples of premium IOLs and other IOLs with fixed optical designs or powers all of which can be used in the base member 102. The power changing lens 104 includes a flexible membrane 402, an optic 406 and an outer circumference 409, which may be referred to as a circumferential peripheral edge. The outer circumference 409 couples the flexible membrane 402 to the optic 406. A membrane coupler 410 is disposed from the outer circumference 409 to couple the flexible membrane 402 with the outer circumference 409. Similarly, an optic coupler 411 is disposed from outer circumference 409 to couple the optic coupler 411 with the outer circumference 409. Preferably, the optic coupler 411 is angled toward the flexible membrane 402 such that it positions the optic 406 toward the flexible membrane 402.

The structure of the power changing lens 104 is simplified by not requiring any traditional elongate thin haptic structures. Rather the peripheral portion 408 is formed as an annulus. The axisymmetric structure enables the power changing lens 104 to be positioned in any rotational position within the cavity 160 in embodiments without cylinder power on the optic 406. Any rotational position of the power changing lens 104 in the base member 102 will provide uniform compression and such compression will provide uniform power change primarily by changing the shape of the flexible membrane 402. For example, ocular forces exerted by the eye can be uniformly compress the power changing lens such that an average change in optical power along any of transverse axes M1, M2, M3 and M4 depicted in FIG. 2A-1 is within ±25% of a nominal optical power. The power changing lens 104 provides a fluid filled lens with one membrane. The optic 406 is a moving optic. The power changing lens 104 changes power through diametrical compression of the peripheral portion 408 in response to ocular forces. Such forces deflect the flexible membrane 402 as indicated by the dashed line anterior of (above) the solid line position of the flexible membrane 402 in FIG. 4B. The optic 406 also moves in response to compression of the peripheral portion 408 as indicated by the dash line anterior of (above) the optic 406 in FIG. 4B. Without subscribing to any particular theory, the uniformity of the power change can be measured using a bench-top measurement system. The bench-top measurement system can comprise a cylindrical device that can hold the IOL device 100 including the base member 102 and the power changing lens 104 in a compressed state similar to the accommodated state in the eye of the patient. The amount of compressive force applied by the cylindrical device can be sufficient to achieve a power change equivalent to an optical power of 4.0 Diopter in the IOL plane. The power change of the IOL device 100 can be considered to be uniform if the average optical power measured along any of the transverse axes M1, M2, M3 and M4 is between 3.0 Diopter and 5.0 Diopter in the IOL plane.

The optic 406 is not a major or main driving force in the change in shape of the flexible membrane 402. Rather, the optic 406 follows the movement of the flexible membrane 402 in response to shifting of the fluid in the closed cavity 412. The optic 406 can be considered to be floating on the fluid in the closed cavity 412 and thus anterior movement of the fluid in response to ocular forces causing compression of the peripheral portion 408 as indicated by arrows A allows the optic 406 to shift anteriorly. Posterior movement of the fluid in response to relaxation of the peripheral portion 408 as indicated by removal of ocular forces in a direction opposite arrows A allows the optic 406 to shift posteriorly. The shifting of the fluid and the optic 406 minimizes distortion of the power changing lens 104 and thus minimizes any dysphotopsia and any other optical interference during power change. The arrows shown within the cross-section in FIG. 4B are intended to show the compression force F divided into components in the power changing lens 104. The vast majority of the force F is driven into the flexible membrane 402 due to the membrane being in the plane of the equator contact segments 141 in the posterior segment 163 of the haptic 124. This is due to the deep set position of the power changing lens 104 in the base member 102. Some force may be transferred into the optic coupler 411. However, a response to this force can be articulating the coupler rather than directly moving the optic 406 forwardly. Thus even the force distribution within the power changing lens 104 attenuates anterior movement driven in response to the compression force F.

The configuration of the power changing lens 104 to enable the optic 406 to follow anterior shape change of the flexible membrane 402 enables the posterior surface of the optic 406 to be placed adjacent to the anterior surface 122 of the base lens 120. The distance between these structures can be 0.5 mm or less, can be 0.4 mm or less, can be 0.3 mm or less, can be about 0.2 mm, or can be 0.2 mm or less. The close positioning of these structures enables the deep inset position of the power changing lens 104 in the base member 102.

In some embodiment the performance of the power changing lens 104 is dependent on placing the power changing lens 104 in the eye such that the flexible membrane 402 is anterior of the optic 406. Also, the manner in which the power changing lens 104 is compressed for insertion into the eye can be critical to successful delivery into the eye. Certain variants aid quickly confirming the orientation of the power changing lens 104.

FIGS. 4A-4B show an optional additional visible color structure 409 that can provide confirmation of the orientation of the power changing lens 104, e.g., to positively identify the location of the flexible membrane 402 and the optic 406. The power changing lens 104 can have a visible color structure 409 disposed in the peripheral portion 408. The visible color structure 409 has an at least partially opaque dye or pigment. The opaque dye or pigment can be any color, which can include red, orange, yellow, green, blue, indigo, violet, and/or any other suitable color or combination of colors. The visible color structure 409 can be a variety of cross-sectional sizes and shapes, which can be continuous or varied. For example, the visible color structure 409 can be a complete annulus that is visible from a peripheral, an anterior and/or a posterior side. The visible color structure 409 can include one or a plurality of arcs or arc segments visible from a peripheral, an anterior and/or a posterior side. The visible color structure 409 is disposed between an anterior portion and posterior portion of the peripheral portion 408 such that the at least partially opaque dye or pigment of the visible color structure 409 is contained in the power changing lens 104 and positioned radially outward of an optical axis A and in some cases outward of a closed cavity 412 of the lens 104. The visible color structure 409 is disposed between a first side 400 (anterior side) and a second side 404 (posterior side) of the power changing lens 104. The visible color structure 409 is disposed closer to the posterior portion than to the anterior portion of the peripheral portion 408 in one example. This positioning enables convenient visual verification of the orientation of the power changing lens 104. The visible color structure 409 is positioned closer to a plane tangential to the posterior surface of the optic 406 than to a plane tangential to an anterior surface of the flexible membrane 402. Accordingly, when viewed from the side, the side of the power changing lens 104 that is closest to the visible color structure 409 is the side of the optic 406, e.g., the second side 404, while the side that is farthest from the visible color structure 409 is the side of the flexible membrane 402, e.g., the first side 400.

The visible color structure 409 can provide a visual verification that the power changing lens 104 is loaded correctly into a injector 480, as seen in FIG. 11. For example, the visible color structure 409 can visually indicate that the power changing lens 104 is loaded into the injector 480 with the flexible membrane 402 folded onto itself such that the flexible membrane 402 is protected from damage and that the power changing lens 104 will exit the injector 480 with the first side 400, e.g., the flexible membrane 402 facing up. Alternatively, the visible color structure 409 can be disposed closer to the anterior portion than to the posterior portion of the peripheral portion 408.

In some aspects, the visible color structure 409 can be used to visually verify that the power changing lens 104 is secured within the base member 102 by the lens retention portions 164. For example, the visible color structure 409 can be a continuous annular shape that is visually disrupted, when viewed from above, by the lens retention portions 164 (if the lens retention portions 164 are opaque or have a solid color, as described elsewhere herein) when the power changing lens 104 is secured within the base member 102 by the lens retention portions 164. Accordingly, the power changing lens 104 is secured by a given lens retention portion 164 when the visible color structure 409 is disrupted, when viewed from above, at the position of the given lens retention portion 164. Relatedly, the power changing lens 104 is not secured by a given lens retention portion 164 when the visible color structure 409 is not disrupted, when viewed from above, at the position of the given lens retention portion 164.

In some aspects, the visible color structure 409 can be combined with an adhesive that joins the anterior portion and posterior portion of the peripheral portion 408. The adhesive can be the same material as the power changing lens 104 or another suitable material. In some aspects, the visible color structure 409 is rotationally symmetrically disposed about the optical axis A. The visible color structure 409 can be an arcuate band surrounding the optical axis A. In some aspects, the visible color structure 409 reduces observable glare transmitted through the peripheral portion 408. FIGS. 4C-4D show examples of a power changing lens 104A that is similar to the power changing lens 104 except as described differently below. The power changing lens 104A has a rotational position feature 413 that is configured to provide simultaneous confirmation of orientation about at least two axes. The rotational position feature 413 can be disposed on or in a peripheral portion of the power changing lens 104A. In one variation, the rotational feature 413 is a visible mark that is oriented in a first direction upon orienting the first side 400 (anterior side) to face an observer and in a second direction, opposite the first direction, upon orienting the first side 400 (anterior side) to face away from an observer. In some aspects, the rotational position feature 413 includes a first mark disposed on a first side of the first side 400 (anterior side) and a second mark disposed on a second side of the first side 400 (anterior side).

In some aspects, as illustrated in FIGS. 4C-4D, the rotational position feature 413 includes an array of dots that confirm orientation about an axis that is oriented perpendicular to the optical axis A. The array of dots can be configured to confirm orientation. For example, the array of dots can include two sets of three dots on opposite sides of a periphery of a surface of the lens 104A, e.g., with the optical axis A disposed between the two arrays of dots. In FIG. 4C, the rotational position feature 413, the arrays of dots, point in a clockwise direction about the optical axis A, which indicates that the power changing lens 104A is positioned with the first side 400 (anterior side) and flexible membrane 402 facing toward an observer. In FIG. 4D, the rotational position feature 413, the array of dots, point in a counterclockwise direction about the optical axis A, which indicates that the power changing lens 104A is positioned with the first side 400 (anterior side) and flexible membrane 402 facing away from an observer and the second side 404 (posterior side) and optic 406 facing towards the observer. In other embodiments the array of dots can be configured to point counter-clockwise about the optical axis A to indicate that the power changing lens 104A is positioned with the first side 400 (anterior side) and flexible membrane 402 facing toward an observer and the second side 404 (posterior side) and optic 406 facing away from the observer.

The rotational position feature 413, e.g., an array of dots, can be formed on an anterior surface, a posterior surface, or an anterior surface and a posterior surface of a peripheral portion of the flexible membrane 402. The rotational position feature 413, e.g., an array of dots, can be formed on a membrane coupler of the power changing lens 104A. The rotational position feature 413, e.g., an array of dots, can be formed on an anterior surface of the peripheral portion 408 of the power changing lens 104A. The rotational position feature 413, e.g., an array of dots, can be formed on a posterior surface of the peripheral portion 408 of the power changing lens 104A. The rotational position feature 413, e.g., an array of dots, can be formed on an optic coupler 411 of the peripheral portion 408 of the power changing lens 104A. The rotational position feature 413, e.g., an array of dots, can be formed on an anterior surface, a posterior surface, or an anterior surface and a posterior surface of a peripheral portion of the optic 406 of the power changing lens 104A.

In some aspects, as illustrated in FIGS. 4C-4D, the rotational position feature 413 includes an array of dots that confirm orientation about the optical axis A. The rotational position feature 413 can be aligned with a transverse axis relative to the optical axis A. For example, the rotational position feature 413 can be aligned with any of the transverse axes M1, M2, M3 and M4 illustrated in FIG. 2A-1 by rotating the power changing lens 104A until the rotational position feature 413, the two sets of arrays of dots, are positioned along the desired axis. The rotational position features 413 can be applied to any lens with rotationally differentiated optics. The rotational position features 413 can be especially advantageous when positioning a toric lens to correct astigmatism.

Further details of the power changing lens 104 can be found in US20160030161A1, which is incorporated by reference herein in its entirety for all purposes.

### E. Intra-Ocular Assembly Methods and Systems

As discussed above, the accommodating IOL device 100 is configured to be assembled in the eye. This configuration enables the accommodating IOL device 100 to be inserted through smaller incisions than would be possible if the accommodating IOL device 100 were fully pre-assembled.

1. Intra-Ocular Assembly Methods

FIG. 10A shows the eye 50 prior to a surgical procedure to implant the accommodating IOL device 100. Although the crystalline lens 54 is shown with uniform optical properties, the eye 50 may be suffering from cataract clouding the crystalline lens 54. The eye 50 may also be suffering from presbyopia which can result when the crystalline lens 54 has become rigid and lacking flexibility to allow the ciliary muscle 70 to deform the lens via the zonules 66 to change the power of the lens. The accommodating IOL device 100 can treat both of these conditions.

FIG. 10B shows the eye 50 following a capsuolotomy. The capsulotomy can start by forming an opening 58 in the front of the crystalline lens 54. The opening 58 is sometimes referred to as a capsulorhexis and can be formed by a scalpel, by a femtosecond laser system or by other techniques. Thereafter the internal volume of the crystalline lens 54 is removed, leaving a sac-like structure referred to herein as the capsular bag 62 The contents can be removed by phacoemulsification or by femto-second laser or by other techniques.

FIG. 10C shows the base member 102 being inserted into the capsular bag 62. The base member 102 is highly compressed by virtue of the configuration of the hinges and undulating structure of the haptic 124 at and between the outer periphery 140. The base member 102 is highly compressed by virtue of the configuration of the hinges between the inner periphery 144 and the ring 292. The enhanced flexibility for compression enables the base member 102 to be inserted through an incision I1 less than or equal to about 3.0 mm. For example, a size of the incision I1 through which the base member 102 can be inserted can be less than or equal to about 2.7 mm, less than or equal to about 2.5 mm, less than or equal to about 2.2 mm and greater than about 1.8 mm. The base member 102 is compressed by folding about a transverse axis TA, e.g., an axis that is perpendicular to the optical axis OA (see FIG. 3C). The base member 102 can be folded such that opposing portions of the haptic 124 at the first edge 152 of the haptic 124 are brought together. The opposing portions of the haptic 124 may be touching each other, as shown in FIG. 10C. In one embodiment, the base member 102 is rolled such that a posterior aspect of the haptic 124 is tucked between an anterior aspect of the haptic 124 and a central area of the fold. After the base member 102 has been folded and/or rolled, it can be coupled with or disposed in an injector 480, shown in part and discussed below in connection with FIG. 11. The injector 480 includes an injector barrel 484 and a plunger 492. A plunging force aligned with the longitudinal axis 500 can push the rolled and/or folded base member 102 into the capsular bag 62.

FIG. 10D shows that the rolled and/or folded base member 102 is advanced out of an injector 480 into the capsular bag 62 with the cavity 160 facing anteriorly. This position is not required but advantageously enables the base member 102 to unfold with the cavity 160 facing anteriorly such that the power changing lens 104 can be inserted without having to inverted the base member 102 to cause the cavity 160 to face anteriorly. The base lens 120 is unfolded such that the equator contact segments 141 are disposed in the equatorial region 74 of the capsular bag 62. Once so positioned the orientation of the base member 102 can be confirmed if the base lens 120 has cylinder power or is otherwise configured to provide optimal optics in one or over a small range of angular positions. As discussed above, the lens retention portion 164A can be configured to visual cue the surgeon as to the orientation of a meridian, diameter or other region with a preferred rotational orientation. Other orientation confirming visual cues or indicia can be used, for example an arrow pointing toward the lens retention portion 164 to be aligned to or disposed opposite of (or at a different position) relative to the incision I1.

FIG. 10E shows the base member 102 fully expanded, e.g. unfolded and/or unrolled. The cavity 160 is facing anteriorly such that another device can be positioned therein. If the rotational orientation of the base member 102 is not as intended, e.g., a cylinder power of the base lens 120 is not in the planned position the method can include rotationally orienting the base member 102 as indicated by the arrow B. The rotation according to the arrow B can be in a shortest arc to provide the proper orientation.

FIG. 10F shows that after the base member 102 has been unfolded and/or unrolled in the capsular bag 62 the power changing lens 104 can be delivered. Preferably the power changing lens 104 is delivered through the same incision, although in one embodiment the power changing lens 104 is delivered through a second incision I2 disposed 180 degrees away from the incision through which the base member 102 is placed. The power changing lens 104 is shown folded or rolled in an opposite orientation to that of the base member 102. FIG. 10E-1 shows that the second side 404 is folded into an interior region of the fold. The power changing lens 104 when folded or rolled is then oriented such that the posterior surfaced (the second side 404) is oriented posteriorly (i.e., rotated 90 degrees from the orientation shown in FIG. 10E-1). When placed in the cavity 160 the power changing lens 104 can unfold with the outer circumference 409 of the peripheral portion 408 extending along the posterior segment 163 of the haptic 124 into position under the lens retention portions 164 and on top of the support surfaces 170.

FIGS. 10E-1 and 10E-2 show various techniques for inserting the power changing lens 104 in simplified schematics of the base member 102 and the power changing lens 104. FIG. 10E-1 shows the power changing lens 104 folded or rolled as described in connection with FIG. 10F. A concave side of a fold or roll would be oriented facing anteriorly (90 degrees into the page). In this configuration the first side 400 of the power changing lens 104 (e.g., the flexible membrane 402) is on the outside of the fold or roll and the optic 406 is on the inside of the fold or roll. The orientation of the power changing lens 104 can be visually verified with the visible color structure 409, which is described in more detail in reference to FIGS. 4A and 4B, to correctly load the power changing lens 104 into the injector 480. As illustrated in FIG. 10E-1, the visible color structure 409 is positioned between the first side 400 and the second side 404 and closer to the second side 404 than the first side 400. The visible color structure 409 visually indicates that the second side 404 and the optic 406 are on the inside of the fold or roll while the first side 400 and the flexible membrane 402 are on the outside of the fold or roll when loading the power changing lens 104 into the injector 480. The base member 102 is oriented in the capsular bag 62 (by motion along the arrows B if needed) such that one of the projection 344 is aligned with an incision I1. Thereafter the rolled or folded power changing lens 104 is inserted directly over the projection 344 that is aligned with the incision I1. Once the power changing lens 104 crosses over the projection 344 that is aligned with the incision I1 the power changing lens 104 is partly advanced out of the injector 480 (as in FIG. 10G) such that a leading edge of the power changing lens 104 upon insertion can be unfolded and/or unrolled and advanced under the other two projections 344 prior to full release of the power changing lens 104 in the base member 102.

FIG. 10E-2 shows a different approach. The power changing lens 104 is folded or rolled and advance over into the eye in an incision I2 that is opposite to one of the projection 344. The incision I2 is disposed between, e.g., equally spaced from the other two projections 344 of the lens retention portion 164. The relative position of the projections 344 to the incision I2 can be achieved by movement of the base member 102 according to the arrow B. The incision I2 is shown opposite to the position of the incision I1 but in general the incisions I1 and I2 can be in the same location or any suitable position. The position can be driven by other factors such as ease of access or corneal contributions to refraction or to higher order aberrations. In the approach of FIG. 10E-2 the power changing lens 104 can be folded or rolled in the opposite direction such that the first side 400 (e.g., the flexible membrane 402) is on the concave or inside of the fold or roll. The second side 404 (e.g., the optic 406) is on the convex or outside of the fold or roll. As illustrated in FIG. 10E-2, the visible color structure 409 is positioned between the first side 400 and the second side 404 and closer to the second side 404 than the first side 400. Accordingly, the visible color structure 409 visually indicates that the first side 400 and the flexible membrane 402 are on the inside of the fold or roll while the second side 404 and the optic 406 are on the outside of the fold or roll when loading the power changing lens 104 into the injector 480. Positioning the first side 400 and the flexible membrane 402 on the inside of the fold or roll can advantageously protect the flexible membrane 402 from damage upon entering or exiting the injector 480. The power changing lens 104 is rotated 90 degrees out of the page and advanced into the eye and partially advanced out of the injector 480 in the cavity 160. The periphery of the power changing lens 104 opposite the injector 480 is slid under the projection 344 opposite the incision I2. Thereafter the power changing lens 104 is further advanced out of the injector until portions of the circumference of the peripheral portion 408 adjacent to the projection 344 are exposed. Clockwise and counter clockwise rotations can allow the peripheral portion 408, e.g., the outer circumference 409, of the power changing lens 104 to move between the projections 344 and the corresponding support surfaces 170.

FIG. 10H shows that gradual expansion of the power changing lens 104 in to the secured position in the posterior segment 163 of the cavity 160 of the haptic 124 as can occur with either the method of FIG. 10E-1 or the method of FIG. 10E-2. A fully extended and secured power changing lens 104 is shown in FIG. 1. When assembled, a minimum distance 295 between a rearward surface of the optic coupler 411 facing the base lens 120 and an upper surface of the ring 292 surrounding the base lens 120 can be less than or equal to about 0.2 mm. For example, the minimum distance 295 between the rearward surface of the optic coupler 411 and the upper surface of the ring 292 surrounding the base lens 120, as indicated at the arrows labeled G in FIG. 2C, can be greater than or equal to 0 mm and less than or equal to 0.05 mm, greater than or equal to 0.02 mm and less than or equal to 0.07 mm, greater than or equal to 0.05 mm and less than or equal to 0.09 mm, greater than or equal to 0.1 mm and less than or equal to 0.15 mm, greater than or equal to 0.12 mm and less than or equal to 0.2 mm, or any value in any range/sub-range defined by these values. Reducing the minimum distance 295 between the rearward surface of the optic coupler 411 facing the base lens 120 and the upper surface of the ring 292 surrounding the base lens 120 can advantageously reduce or prevent migration of cells through the channels 109 thereby reducing the risk of interlenticular PCO between the base lens 120 and the power changing lens 104. The gap may permit some fluid flow 110 at indicated by dashed arrows but generally limit, reduce or prevent cell migration. Additionally, reducing the minimum distance 295 between the rearward surface of the optic coupler 411 facing the base lens 120 and the upper surface of the ring 292 surrounding the base lens 120 can advantageously reduce the risk of retinal detachment as a result of filling the capsular bag.

If needed, the power changing lens 104 and/or the projection 344 can be repositioned such that the power changing lens 104 is configured to be properly seated in the posterior segment 163. Such repositioning can follow a visual inspection of the expanded power changing lens 104 and the expanded base member 102. As discussed in connection with FIG. 8 the ridge 372 can give clear visual cues as to whether the retention member 344A is anterior of the first side 400 of the power changing lens 104 or is posterior of the second side 404 thereof. If the retention member 344A is posterior of the power changing lens 104 the surgeon will reposition the peripheral portion 408 in the area of the retention member 344A such that the first side 400 is posterior of the retention member 344A.

Other forms of visual cues or indicia can be provided. For example, the ring 292 and/or other portions of the haptic 124 can be formed with a high contrast color, such as a blue color. One or more portions of, e.g., all of, the haptic 124 including the ring 292 can be formed of the materials discussed herein, including silicone materials, and can include a contrast forming component, such as a blue pigment or dye. For example, some silicone haptic materials may be blended with a color masterbatch material prior to curing, such color masterbatch including, but not limited to, the MED-4800 series from NuSil®, including MED-4800-7 (dark blue). Preferred pigments are substantially non-extractable from the cured material by water, saline or ocular fluids at about 37° C. The position of the ring 292 can be confirmed prior to insertion of the power changing lens 104. The ring 292 can be confirmed to be centered on the visual axis of the eye, for example. The ring 292 can have a portion of a different color or with another visual cue that shows the orientation of a cylinder power. Different patterns can be provided on the membrane coupler 410 and on the anterior surface of the projection 344 of the lens retention portion 164. For example the membrane coupler 410 can have a smooth finish and the anterior surface of the projection 344 can have a dull or matt finish. If there is an unbroken arc of smooth finish of more than 120 degrees the projection 344 can be confirmed to be posterior to the peripheral portion 408 of the power changing lens 104. Instead of smooth and matt finishes, one of the peripheral portion 408 and the projection 344 can have hatching in one direction and the other can have hatching in another direction. Or, one of the peripheral portion 408 and the projection 344 can have a first color surface and the other can have a second color visually distinct from the first color.

FIG. 10I illustrates the power changing lens 104A positioned within the base member 102 and the eye 50. As described in reference to FIGS. 4C and 4D, the rotational position feature 413 can be used to simultaneously verify orientation of the power changing lens 104A about the optical axis and an axis perpendicular to the optical axis. For example, the array of dots of the rotational position feature 413 can be seen as an arrow that points in a clockwise direction indicating that the power changing lens 104A is orientated with the first side 400 (anterior side) and flexible membrane 402 facing away from the base lens 120, which is the correct orientation. The array of dots of the rotational position feature 413 also can visually indicate that the rotational position feature 413 is rotationally aligned about the optical axis to correspond to a transverse axis that falls along a prescribed orientation (e.g., 12 to 6 of a clock face) to match the unaccommodated power of the lens 104A along that axis to the visual deficiency of the eye, e.g., for correction of astigmatism. If needed, the array of dots of the rotational position feature 413 can visually indicate an orientation corresponding to any perpendicular axis relative to the optical axis, such as a 2 to 8, 4 to 10, 5 to 11, or any other orientation (referring to a clock face to describe the transverse axes) as the power changing lens 104A is rotated. As described above, the rotational position feature 413 can be a applied to a toric lens for confirming rotational positioning about the optical axis and can be beneficial for all types of lenses to confirm anterior-posterior orientation.

2. Systems for Intra-Ocular Assembly

FIG. 11 shows a portion of the injector 480, which has been discussed above. The injector 480 includes the injector barrel 484. The injector barrel 484 has a barrel tip 488 including an opening through which one or both of the base member 102 and the power changing lens 104 are injected into the eye 50.

A inner barrel 504 can be located in the injector barrel 484. The inner barrel 504 can house the power changing lens 104 in one embodiment. The inner barrel 504 can be centered on the longitudinal axis 500 and can be moveable along the longitudinal axis 500 independently of or relative to the injector barrel 484. For example, the inner barrel tip 508 can be initially proximal of the barrel tip 488. The inner barrel tip 508 can be advanced out of the barrel tip 488 during part of a procedure.

The 580 can include a plunger 492 that, as discussed above can be used to advance one or both of the base member 102 an the power changing lens 104 out of the injector 480. The plunger 492 can include a plunger tip 496 that can be disposed proximal to one or both of the base member 102 and the power changing lens 104. The plunger tip 496 can push the base member 102 or the power changing lens 104 out of the injector 480 into the eye.

In one approach the injector barrel 484 and the inner barrel 504 are separate instruments that can be mounted to the same or to different handles. The injector barrel 484 can have a proximal end that is mounted to such a handle and can be inserted through the incision I1 or the incision I2 to place the base member 102. After the plunger 492 advances the base member 102 out of the injector 480 into the eye 50 the injector barrel 484 can be removed from the handle and the inner barrel 504 can be attached to the handle. When the injector barrel 484 and the inner barrel 504 are separate the inner barrel 504 is an inner barrel in that it delivers the inner device, e.g., the power changing lens 104. The plunger 492 can be configured to be used with both the injector barrel 484 and thereafter with the inner barrel 504. In other words, the plunger 492 can be part of the handle and can push both the base member 102 out of the injector 480 and can push the power changing lens 104 out of the inner barrel 504 after the injector barrel 484 has been removed and the inner barrel 504 mounted on the handle and the plunger 492 of the injector 480.

In another approach the inner barrel 504 is mounted within the injector barrel 484. The plunger 492 or the inner barrel 504 can be used to push the base member 102 out of the barrel tip 488. Thereafter the plunger 492 and the inner barrel 504 can be advanced toward and in some cases out of the barrel tip 488. The power changing lens 104 can then be urged by the plunger 492 out of the barrel tip 488.

FIG. 11 shows one approach in which the base member 102 and the power changing lens 104 are both folded such that the concave portions of the folded structure are facing the same direction. The open side of the folded structure can be oriented in the injector 480 such that the open side faces anteriorly. This is advantageous for the method illustrated in FIG. 10E-1.

In another embodiment the base member 102 and the power changing lens 104 are folded and loaded in the injector 480 such that the concave portions of the folded structure face in opposite directions. The open side of the folded structure of the base member 102 can be oriented in the injector 480 such that the open side faces anteriorly. The open side of the folded structure of the base member 102 can be oriented in the injector 480 such that the open side faces posteriorly (as in the dashed lines). This is advantageous for the method illustrated in FIG. 10E-2.

The lumens of one or both of the injector barrel 484 and the inner barrel 504 can be treated to provide advantageous performance as taught by U.S. Pat. No. 7,037,312 which is hereby incorporated by reference herein in its entirety. For example, the lumen just proximal to the barrel tip 488 can be configured to gradually expand the base member 102. In one approach the lumen just proximal to the barrel tip 488 is expandable to slowly expand the base member 102 prior to the base member 102 being fully expelled from the barrel tip 488. In another approach the coefficient of friction of the lumen just proximal to the barrel tip 488 can be higher such that the injector naturally slows the egress of the base member 102. In yet another approach the inner diameter of the injector barrel 484 can be reduced to elongate the base member 102 which in turn can reduce the speed at which the base member 102 is released. Similar approaches can be used for the inner barrel 504.

As discussed in greater detail herein the base member 102 is configured to be inserted into the eye 50 through a small incision. This is facilitated by a number of hinge connections discussed below and by a reduction in material of the base member 102 where not needed.

### II. Advantages and Other Applications of Two-Part Configurations

The accommodating IOL device 100 is modular which includes the idea that the base member 102 and the power changing lens 104 can be assembled separately. In one technique, separate assembly enables the base member 102 to be implanted first and the power changing lens 104 to be implanted subsequently, as discussed further below. Where the power changing lens 104 is assembled forward of the base member 102, the power changing lens 104 also can be removed and replaced during the same procedure or in a subsequent procedure. Same procedure replacement can facilitate adjustment of the unaccommodated (e.g., distance) power. Such adjustment can enable a surgeon to address a surgical complication where the actual placement of the lens is different form the planned location of the lens and the difference causes the unaccommodated power to be too strong or too weak. This issue can be assessed at the time of the procedure by intraoperative assessment, such as using aberrometry. If the power is measured as too low, the initial power changing lens 104 can be exchanged for a power changing lens 104 with a higher unaccommodated power. If the power is measured as too high, the initial power changing lens 104 can be exchanged for a power changing lens 104 with a lower unaccommodated power.

In some cases, a difference arises between planned placement and post-procedure locations, e.g., after a period of recovery. The process of recovery can cause the base member 102 to shift from a planned or intraoperative position. If the shift is large enough even a perfect intraoperatively measured unaccommodated power can become noticeably too high or too low. After the recovery period, the initially placed power changing lens 104 can be removed and replaced with a power changing lens 104 having a power selected based on the final, healed position of the base member 102. Without the ability to exchange the initially placed power changing lens 104 for a power changing lens 104 with more appropriate power, a larger number of patients would not achieve spectacle independence.

The separateness of the base member 102 and the power changing lens 104 provides additional benefits even when the base member 102 and power changing lens 104 are perfectly placed and matched. For instance, while the power changing lens 104 described below has been shown to provide excellent range of accommodation, improvements in design or changes in the patient's vision may make a lens upgrade advantageous. The power changing lens 104 can be removed and replaced with a higher performance lens or with a lens configured to address particular and in some cases changed vision needs of the patient.

Furthermore, the base member 102 provides a support for implanting second lenses that may not be fluid lenses. Some patients may prefer not to receive a biomimetic accommodating lens, such as with the power changing lenses described herein, but may prefer other types of lenses. The base member 102 can be implanted in such patients to enable such patients to receive a higher performance lens at a later date. For example, FIGS. 4E-4F show examples of lenses that can be combined with the base member 102 to provide excellent outcomes short of biomimetic accommodation.

FIG. 4E shows a monofocal lens 104B. The monofocal lens 104B has a fixed optic 403B, e.g., one that does not accommodate as described in reference to power changing lens 104. The fixed optic 403B has a set power that does not change in response to ocular forces. The monofocal lens 104B can have a monolithic structure having a continuous body between an anterior surface and posterior surface of the monofocal lens 104B and/or fixed optic 403B. The monolithic structure enables the monofocal lens 104B to be manufactured with a single molding step. In some aspects, the monofocal lens 104B can be a monofocal toric lens to correct astigmatism. The fixed optic 403B can have different optical powers along different perpendicular axes relative to the optical axis A, sometimes called toric axes, when the monofocal lens 104B is also a toric lens. In other cases the monofocal lens 104B has rotationally symmetrical optics.

The monofocal lens 104B has a circular haptic 401B. The circular haptic 401B is centered around the optical axis A. The circular haptic 401B is radially outward of an optical axis A. The circular haptic 401B surrounds the fixed optic 403B. The circular haptic 401B can be placed under the lens retention portions 164 of the base member 102 to secure the monofocal lens 104B to the base member 102, such as shown in FIG. 10H. The monofocal lens 104B can have, as described in reference to FIGS. 4A and 4B, a visible color structure 405B. The visible color structure 405B can be the same as or similar to the visible color structure 409 discussed above. The visible color structure 405B can be positioned in the periphery of the monofocal lens 104B, such as in or on the circular haptic 401B. The visible color structure 405B can be positioned on an anterior surface of the circular haptic 401B, posterior surface of the circular haptic 401B, or between the anterior and posterior surface of the circular haptic 401B, e.g., within the anterior-posterior thickness thereof. The visible color structure 405B can be insert molded into the fixed optic 403B or can be molded onto or into the circular haptic 401B. The visible color structure 405B can be used to visually verify orientation of the monofocal lens 104B as described in reference to FIGS. 4A and 4B. The visible color structure 405B can be used to visually verify that the monofocal lens 104B is secured by the lens retention portions 164 when monofocal lens 104B is being positioned within the base member 102 as described in reference to FIGS. 4A and 4B. In some aspects, the visible color structure 405B reduces observable glare transmitted through the circular haptic 401B, such as by one or more of reflecting, absorbing, or diffusing stray light.

The monofocal lens 104B can have a rotational position feature 413, that is the same as or similar to the rotational position feature 413 described in reference to FIGS. 4C and 4D. The rotational position feature can be used to provide simultaneous confirmation of orientation about at least two axes, indicating rotational orientation about the optical axis A and rotational orientation about an axis that is perpendicular to the optical axis A. When the monofocal lens 104B is a toric lens, the rotational orientation features 413 can be aligned with a specific transverse axis relative to the optical axis A, such as a toric axis, to properly orient the toric lens to correct astigmatism. For example, as described above, the rotational position feature 413 can visually indicate an orientation corresponding to any perpendicular axis relative to the optical axis A, such as a 2 to 8, 4 to 10, 5 to 11, or any other orientation (referring to a clock face to describe the transverse axes) as the monofocal lens 104B is rotated about the optical axis A.

FIG. 4F shows a fixed multi-focal or multi-powered lens 104C. The fixed multi-powered lens 104C has a fixed optic 403C that does not accommodate as described in reference to power changing lens 104. The fixed multi-powered lens 104C can have a monolithic structure having a continuous body between an anterior surface and posterior surface of the fixed multi-powered lens 104C and/or fixed optic 403C. The monolithic structure enables the fixed multi-powered lens 104C to be manufactured with a single molding step. In some aspects, the fixed multi-powered lens 104C can be a bifocal or trifocal lens, providing respectively two or three fixed focal points. In some aspects, the fixed multi-powered lens 104C can be an extended depth of focus (EDOF or EDF) lens, having an elongated focal point to provide a range of powers.

The fixed multi-powered lens 104C has a circular haptic 401C. The circular haptic 401C can be the same or similar to the circular haptic 401B. The circular haptic 401C can be placed under the lens retention portions 164 of the base member 102 to secure the fixed multi-powered lens 104C to the base member 102, such as shown in FIG. 10H. The fixed multi-powered lens 104C can have, as described in reference to FIGS. 4A and 4B, a visible color structure. The visible color structure can be the same as or similar to the other visible color structures described herein. The visible color structure can be used for orientating the fixed multi-powered lens 104C as described herein. The fixed multi-powered lens 104C can have a rotational position feature, that is the same as or similar to the other rotational position features described herein. The rotational position feature can be used to provide simultaneous confirmation of orientation about at least two axes, indicating rotational orientation about the optical axis A and rotational orientation about an axis that is perpendicular to the optical axis A.

The circular haptics 401B, 401C enable the lenses 104B, 104C to be compatible with a base member 102 that is configured to apply rotationally symmetrical compression to a power changing lens with a circular haptic when the base member is subject to rotationally symmetric compression, e.g., in the eye or in a bench-top test. The base member 102 is thus compatible with fixed optics such as the lenses 104B, 104C and can be upgraded to an accommodating IOL such as the power changing lenses 104, 104A or another biomimetic IOL. By providing circular haptics the lenses 104, 104A, 104B, 104C can be implanted in any rotational position within the base member 102 if a specific rotational position is not indicated. A circular haptic can facilitate re-positioning in the eye where a specific rotational position is indicated. For example, the lens 104A can be situated in the base member 102 posterior of the tabs or other lens retention portion 164. If the rotational position is to be adjusted, the circular haptics allow for the lens 104A to be rotated in position, with the rotational position features 413 aligned with a specified axis.

### Terminology

As used herein, the relative terms “proximal” and “distal” shall be defined from the perspective of the medical professional. Thus, proximal refers to the direction of the physician and distal refers to the direction of the eye when the surgeon is operating.

For expository purposes, the term “transverse” as used herein is defined as a direction generally perpendicular to the longitudinal axis of the assembly, unless otherwise specified.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The terms “approximately,” “about,” “generally,” and “substantially” as used herein represent an amount close to the stated amount that still performs a desired function or achieves a desired result. For example, the terms “approximately,” “about,” “generally,” and “substantially” may refer to an amount that is within less than 10% of the stated amount, as the context may dictate.

The ranges disclosed herein also encompass any and all overlap, sub-ranges, and combinations thereof. Language such as “up to,” “at least,” “greater than,” “less than,” “between” and the like includes the number recited. Numbers preceded by a term such as “about” or “approximately” include the recited numbers. For example, “about four” includes “four”

Any methods disclosed herein need not be performed in the order recited. The methods disclosed herein include certain actions taken by a practitioner; however, they can also include any third-party instruction of those actions, either expressly or by implication. For example, actions such as “distally moving a locking element” include “instructing distal movement of the locking element.”

Although certain embodiments and examples have been described herein, it will be understood by those skilled in the art that many aspects of the humeral assemblies shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

What is claimed is:

1. An intraocular lens component, comprising:
an anterior side comprising an anterior optical surface disposed across an optical axis of the lens component;
a posterior side comprising a posterior optical surface disposed across the optical axis;
a peripheral portion having an anterior portion coupled to the anterior side and a posterior portion coupled to the posterior side, the peripheral portion coupling the anterior side to the posterior side of the intraocular lens component; and
a visible color structure disposed in the peripheral portion between the anterior portion and the posterior portion thereof.

2. The intraocular lens component of claim 1, wherein the visible color structure comprises an at least partially opaque dye or pigment.

3. The intraocular lens component of claim 2, wherein the at least partially opaque dye is contained in the intraocular lens and is positioned radially outward of the optical axis.

4. The intraocular lens component of claim 1, wherein the visible color structure is disposed between the anterior side and the posterior side.

5. The intraocular lens component of claim 1, wherein the visible color structure is disposed closer to the anterior portion than to the posterior portion of the peripheral portion or closer to the posterior portion than to the anterior portion of the of the peripheral portion.

6. The intraocular lens component of claim 1, further comprising a closed cavity disposed between the anterior side, the posterior side and the peripheral portion, the closed cavity configured to enclose an optical fluid between the anterior optical surface and the posterior optical surface along the optical axis.

7. The intraocular lens component of claim 6, wherein the visible color structure comprises an adhesive and wherein the anterior portion of the peripheral portion and the posterior portion of the peripheral portion are joined by the visible color structure.

8. The intraocular lens component of claim 1, wherein the visible color structure is rotationally symmetrically disposed about the optical axis.

9. The intraocular lens component of claim 8, wherein the visible color structure comprises an arcuate band surrounding the optical axis.

10. The intraocular lens component of claim 1, further comprising a continuous body between the anterior optical surface and the posterior optical surface.

11. The intraocular lens component of claim 10, wherein the continuous body comprises a monofocal lens.

12. The intraocular lens component of claim 10, wherein the continuous body comprises a toric lens.

13. The intraocular lens component of claim 10, wherein the continuous body comprises a multifocal lens.

14. The intraocular lens component of claim 1, wherein visible color structure is configured to reduce observable glare transmitted through the peripheral portion.

15. An intraocular lens component, comprising:
an anterior side comprising an anterior optical surface disposed across an optical axis of the lens component;
a posterior side comprising a posterior optical surface disposed across the optical axis;
a peripheral portion having an anterior portion coupled to the anterior side and a posterior portion coupled to the posterior side, the peripheral portion coupling the anterior side to the posterior side of the intraocular lens component; and
a rotational position feature disposed on or in the peripheral portion configured to provide simultaneous confirmation of orientation about at least two axes.

16. The intraocular lens component of claim 15, wherein the rotational position feature comprises a visible mark configured to be oriented in a first direction upon orienting the anterior side to face an observer and in a second direction opposite the first direction upon orienting the anterior side to face away from the observer.

17. The intraocular lens component of claim 15, wherein the rotational position feature comprises a first mark disposed on a first side of the anterior optical surface and a second mark disposed on a second side of the anterior optical surface, the second mark disposed opposite the first mark.

18. The intraocular lens component of claim 15, wherein the rotational position feature comprises an array of dots configured to indicate a direction confirming orientation about an axis oriented perpendicular to the optical axis.

19. A method of assembling a base member of an intraocular lens, comprising:
providing a base member haptic comprising a first open end, a second end opposite the first open end, and an outer periphery configured to engage an equatorial region of a capsular bag, the second end having a lens interface portion;
providing a base lens having a central optical portion and a peripheral haptic interface portion;
coupling the haptic interface portion of the base lens with the lens interface portion of the base member haptic; and
securing the base lens and the base member haptic together at the lens interface portion and the haptic interface portion.

20. A method of assembling an intraocular lens in a capsular bag of an eye of a patient, comprising:
positioning a bowl-shaped member in a capsular bag of an eye with a base lens of the bowl shaped member contacting a posterior inside surface of the capsular bag and a haptic contacting an equatorial region of the capsular bag, the haptic having a first edge disposed forward of an anterior surface of the base lens, the bowl-shaped member defining a cavity therein;
advancing a power changing lens into the cavity of the bowl shaped member in a folded state wherein opposing sides of a circumferential portion of the power changing lens are brought together; and
unfolding the power changing lens within the cavity of the bowl-shaped base member such that the circumferential portion of the power changing lens is retained within the haptic.
